(19) United States
(12) Patent Application Publication
(10) Pub. No.: US 2017/0067840 A1
(43) Pub. Date: Mar. 9, 2017

(54) DIAGNOSTIC DEVICE FOR EVALUATING MICROBIAL CONTENT OF A SAMPLE

(71) Applicant: Telemedicine Up Close, Inc., dba DxUpClose, Frisco, TX (US)

(72) Inventors: Wayne T. Kilian, Telephone, TX (US); James R. Biard, Richardson, TX (US); Jay B. Nickel, Wimberley, TX (US); Gregory C. Roach, Frisco, TX (US); Keith T. Rommel, Lewisville, TX (US); Cynthia S. Nickel, Wimberley, TX (US)

(21) Appl. No.: 14/845,027

(22) Filed: Sep. 3, 2015

(51) Int. Cl. G01N 27/04; C12Q 1/06; C12Q 1/18; C12Q 1/04

(52) U.S. Cl. CPC G01N 27/041; C12Q 1/04; C12Q 1/06; C12Q 1/18

(57) ABSTRACT

A diagnostic device evaluates microbial content of a sample. In some embodiments, the diagnostic device includes a plurality of sample cells in which the microbial content of a sample is evaluated. Electronic circuitry is used to apply electrical signals to electrodes that interact with the sample in the sample cells. The electronic circuitry also measures one or more characteristics of the sample. Using the measured characteristics, the diagnostic device performs one or more of: identifying microbes, counting microbes, and determining antimicrobial sensitivity of microbes within the sample.

### BACKGROUND

One of the difficulties encountered in the treatment of issues involving microbes is that microbes are continually changing. Microbes include, for example, bacteria, fungi, viruses, nematodes, cell culture, and tissue. Microbes are becoming antimicrobial resistant. Antimicrobials include antibiotics, antivirals, antifungals, or parasiticides and include bacteriophage, mycoviruses, virophages, nematophages, which are viruses that attack bacteria, fungi, nematodes, respectively. With the rise of antibiotic resistant bacteria, for example, even if the type of bacteria is properly identified, a prior treatment thought to be effective against such bacteria may no longer be so. More specifically, the particular species of bacteria may have developed a resistance to the treatment, and therefore no longer be susceptible to that treatment.

### SUMMARY

In general terms, this disclosure is directed to a diagnostic device that evaluates microbial content of a sample. In one possible configuration and by non-limiting example, the diagnostic device performs one or more of: determining antimicrobial sensitivity of microbes, identifying microbes, and counting microbes. Various aspects are described in this disclosure, which include, but are not limited to, the following aspects.

One aspect is a diagnostic device comprising: at least one sample module defining a sample cavity therein; at least four electrodes arranged in the sample cavity; and electronic circuitry operably connected to the electrodes, wherein the electronic circuitry is operable in a first mode and a second mode, wherein when operating in the first mode, the electronic circuitry operates to determine a conductance of a sample in the sample cavity, and wherein when operating in the second mode, the electronic circuitry operates to determine an admittance of the sample in the sample cavity.

Another aspect is an antimicrobial dispenser comprising: a sterile carrier material; and bacteriophage carried by the sterile carrier material.

A further aspect is a sample module comprising: at least one substrate; at least four electrodes; and a sample cavity formed in the at least one substrate, the sample cavity comprising: a sensing portion including the electrodes therein, the sensing portion having a shape configured to direct and focus electric fields generated by the electrodes within the sample cavity; and a chimney portion extending from the sensing portion and having a cross-sectional size that is less than a cross-sectional size of the sensing portion.

Yet another aspect is a diagnostic device comprising: a plurality of sample modules; electrodes arranged in the sample modules; a calibration fluid disposed in a calibration module of the sample modules; and electronic components coupled to the electrodes, wherein the electronic components are operable to measure a conductivity of the fluid in the calibration cell and to determine a temperature of the calibration fluid using the measured conductivity.

Additional aspects are disclosed herein.

### BRIEF DESCRIPTION OF THE DRAWINGS

FIG. 1 is schematic perspective view of an example diagnostic device.

FIG. 2 is a block diagram of an example reader of the diagnostic device shown in FIG. 1.

FIG. 3 is a block diagram of an example diagnostic unit of the diagnostic device shown in FIG. 2.

FIG. 4 is a schematic perspective view of an example sensor system of the diagnostic unit shown in FIG. 3.

FIG. 5 is a schematic perspective view of an example sample cell of the sensor system shown in FIG. 4.

FIG. 6 is a cross-sectional side view of the sample cell shown in FIG. 5.

FIG. 7 is a top view of the sample cell shown in FIG. 5, also illustrating an electrode configuration and an example antimicrobial dispenser.

FIG. 8 is a top cross-sectional view of another example sample cell and another example electrode configuration.

FIG. 9 is a top cross-sectional view of another example sample cell and another example electrode configuration.

FIG. 10 is a cross-sectional top view of another example sample cell and another example electrode configuration.

FIG. 11 is a cross-sectional top view of another example sample cell and another example electrode configuration.

FIG. 12 is a cross-sectional side view of the example sample cell and example electrode configuration shown in FIG. 11.

FIG. 13 is a state diagram illustrating an example of the operation of the diagnostic device shown in FIG. 1.

FIG. 14 is a perspective view of an example antimicrobial dispenser.

FIG. 15 is a perspective view of the example antimicrobial dispenser shown in FIG. 14, illustrating the dispensing of the antimicrobial into a fluid.

FIG. 16 is a schematic block diagram illustrating an exemplary architecture of a computing device that can be used to implement aspects of the present disclosure.

FIG. 17 illustrates another exemplary architecture involving a diagnostic device.

FIG. 18 illustrates experimental data obtained using a diagnostic device.

FIG. 19 illustrates additional experimental data obtained using a diagnostic device.

FIG. 20 illustrates additional experimental data obtained using a diagnostic device.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

In general terms, this disclosure is directed to a diagnostic device that evaluates microbial content of a sample. In some embodiments, the diagnostic device performs one or more of: determining antimicrobial sensitivity of microbes, identifying microbes, and counting microbes.

In some embodiments, the diagnostic device provides a rapid (e.g., one hour) antimicrobial sensitivity test. With the increase of antibiotic resistant bacteria, the antimicrobial sensitivity test enables medical professionals to prescribe the correct antibiotic the first time with less opportunity for antimicrobial resistant microbes to evolve. When a patient arrives at an emergency room with signs of sepsis, those patients who have an effective antimicrobial regime started within the first hour have a better outcome than patients who have delayed treatment. Additionally, the diagnostic device can, in some embodiments, identify and/or screen for bacteria that require special protocols (e.g. MRSA, vancomycin resistant bacteria, and other such deadly bacteria), to assist healthcare professionals in identifying and selecting treatment protocols that are effective, while reducing side effects of the treatment. Counting bacteria quantities can also be performed by the diagnostic device to quantify the severity of an infection.

In some embodiments, the diagnostic device operates to analyze microbe samples developed from human or animal samples of blood, urine, sweat, mucus, saliva, semen, vaginal secretion, vomit, tears, sebum, pleural fluid, peritoneal fluid, gastric juice, earwax, cerebrospinal fluid, breast milk, endolymph, perilymph, aqueous humor, vitreous humor, biomass or the like, by measuring one or more electrical characteristics of the sample in the sample cells. Further, some embodiments of the diagnostic device operate to detect harmful microbes on food and to detect harmful corrosive microbes existing in pipelines of sewers, oil, gas and chemical plants, for example.

In embodiments, the microbes are selected from Aerobacter, Bacillus, Bordetella, Brucella, Campylobacter, Chlamydia, Chromobacterium, Clostridium, Corynebacterium, Enterobacter, Escherichia, Haemophilus, Klebsiella, Listeria, Mycobacterium, Mycoplasma, Neisseria, Pneumococcus, Proteus, Pseudomonas, Providencia, Salmonella, Serratia, Shigella, Staphylococcus, Streptococcus, Vibrio, Yersinia, Acinetobacter, Bacteroides, Bifidubacterium, E. kenella corrodens, Gardnerella vaginalis, Mobiluncus, Proteobacteria, Desulfobacterales, Desulfovibrionales, Syntrophobacterales, Thermodesulfobacteria, Nitrospirae, gram-positive Peptococcaceae, Archaea, Archaeoglobus, or any combinations thereof.

For each bacteria type there is a unique viral phage that attacks and kills only that specific bacterium. When bacteria are attacked by specially cultivated bacteriophage, bacteria can be made to release ions as the bacteriophage inject their DNA into the bacteria thus causing an ionic flux that can be detected electronically. In some embodiments, some of the sample cells in the array are loaded with nutrient broth, or with microbes plus nutrient broth, or with different antibiotics with different viral phages or antibiotics with one unique phage or antibiotic in each of at least some of the sample cells. Such bacteriophages can be embedded in a material that allows for controlled elution of the bacteriophage. Since each different viral phage attacks only one specific bacterium, the identity of the bacterium in the test can be determined. In some embodiments the bacterium identity is determined from analyzing the difference in electrical conductivity signatures between the sample cells that contain only nutrient broth; nutrient broth and microbes; and nutrient broth, microbes, and viral phage. In other embodiments the bacterium identity can be determined from a distinct signal generated when the bacteriophage attack their targeted bacteria, which occurs within the first fifteen minutes of the bacteria being introduced to the sample cell with the bacteriophage impregnated material. It is our hypothesis that bacteriophage can be cultivated with long shelf lives while impregnated in such material.

In embodiments, the phages can be selected from Actinomyces phages, Bacillus phage Φ29, bacteriophage M102, bacteriophage e10, bacteriophage f1, bacteriophage λ, bacteriophage PI, spherical phage PhiX174, spherical phage G4, spherical phage S13, bacteriophage T1, bacteriophage T2, bacteriophage T3, bacteriophage T4, bacteriophage T5, bacteriophage T6, bacteriophage T7, ssRNA bacteriophages MS2, ssRNA bacteriophages R17, ssRNA bacteriophages f2, ssRNA bacteriophages Q beta, S. mutans phages, and any combinations thereof

In further embodiments, the antimicrobials can be selected from amikacin, azlocillin, carbencillin, cefaclor, cefemandole, cefonicid, cefotaxime, cefoperazone, cefoxitin, ceftizoxime, ceftriaxzone, ciprofloxacin, clindamycin, gatifloxacin, gemifloxacin, gentamicin, kanamycin, linezolid, mecillinam, meropenem, methicillin, metronidazole, mezlocillin, minocyclin, moxifloxacin, nafcillin, netilmycin, oxacillin, penicillin, piperacillin, quinupristin-dalfopristin, sparfloxacin, sulbactam, tazobactam, teicoplanin, tetracyclines, tobramycin, trimethoprim, trospectomycin and vancomycin.

By performing calibration tests with known concentrations of different microbes, the value of the measured conductivity for sample cells that contain nutrient broth and microbes can be used to assess the concentration of microbes in the sample cell (e.g., count the bacteria in the sample cell).

Antimicrobials, such as antibiotics and bacteriophages, cause the metabolism to slow down or cease, yielding one or more detection mechanisms specific to a given antimicrobial agent's ability to eradicate the microbe. Monitoring the microbes during the first period of time (e.g., one hour) after the antimicrobial attack provides a good indication of the final outcome of the antimicrobial. Other sample cells in the array may be loaded with nutrient broth, microbes, and an array of unique antimicrobial agents in each of a plurality of the sample cells. Antimicrobial agents include antibiotics, antimicrobial peptides, bacteriophage and small molecule drugs, for example. In some embodiments, the effectiveness of different antimicrobial agents in killing the microbes, such as bacteria, fungi, viruses, nematodes, cell culture, or tissue, can be determined by comparing the electrical signal (conductance or admittance) signatures of the nutrient broth only; nutrient broth and microbes; and nutrient broth, microbes, and antimicrobials in the various sample cells. In other embodiments, a distinct digital signal can be associated with each distinct antimicrobial and its positive or negative effect on the microbe's resistance to the distinct antimicrobial, each correlated signal thus becoming a digital signature. Comparison of the digital signature against a database of digital signatures can determine effectivity of the antimicrobial. When bacteria identity can be determined, this information also may optionally be used to determine antibiotic effectivity. This is especially important when β-lactamase producing bacteria, such as staphylococcus spp, are identified or other extended-spectrum beta-lactamase (ESBL) bacteria are detected, for example.

In some embodiments, a mixture of bacteria in a sample can also be detected using the diagnostic device. For example, if a bacterial sample contains two different bacteria, growth of both bacteria will occur in multiple cells but a decrease in growth will be seen in more than one cell due to lysis of each of the different bacteria with a different bacteriophage.

Some embodiments of the diagnostic device utilize analog impedimetric/conductometric measurement instruments and techniques for the detection and quantization of bacteria present in a broth culture. Some embodiments also relate to methods of manufacturing impedimetric measurement vessels.

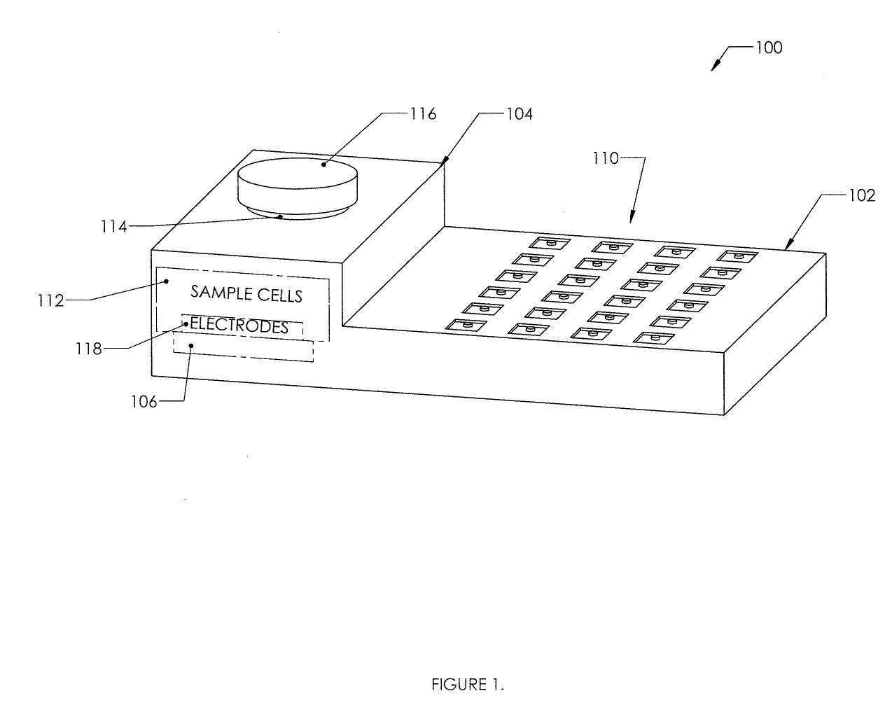

FIG. 1 is schematic perspective view of an example diagnostic device 100. In some embodiments, the diagnostic device includes a reader 102, a diagnostic unit 104, and an interface 106.

The diagnostic device 100 operates to evaluate microbial content of a sample. In some embodiments, the diagnostic device 100 is formed as a single part. However, in other embodiments the diagnostic device 100 is formed as at least two parts, as shown in FIG. 1, including a reader 102 and a diagnostic unit 104. An advantage of forming the diagnostic device 100 of at least two parts is that the reader 102 can be configured as a reusable part, while the diagnostic unit 104 can be configured as a disposable part. The sample is contained entirely within the disposable part, while at least most of the electronics are contained within the reader. The interface 106 permits communication between the reader 102 and the diagnostic unit 104.

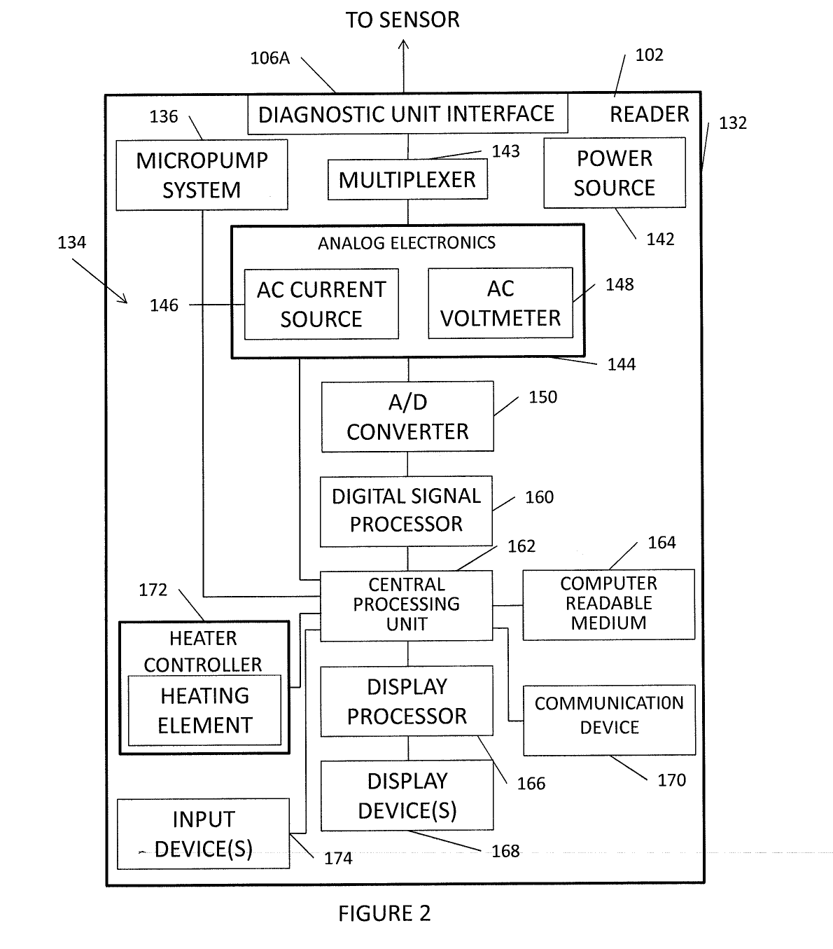

In one example, the reader 102 contains electronic components that operate in conjunction with the diagnostic unit 104 to evaluate microbial content of the sample. In some embodiments, the reader 102 includes analog electronics that generate alternating current (AC) signals that are provided to the diagnostic unit 104 for interrogating the sample. The reader 102 also includes, in some embodiments, sensing electronics for detecting one or more characteristics of the sample during the interrogation to evaluate the microbial content of the sample. Some embodiments also include a display device 110, or other output device, for conveying results of the microbial evaluation performed by the diagnostic device 100. An example of the reader 102 is illustrated and described in more detail with reference to FIG. 2.

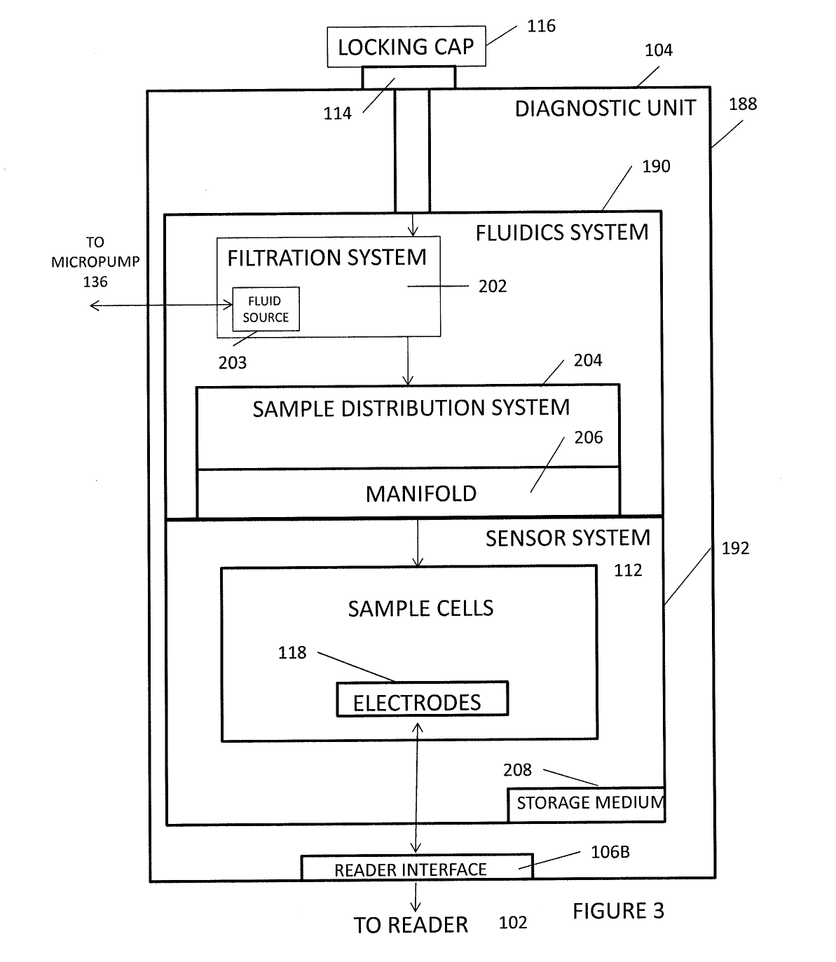

The diagnostic unit 104 includes one or more sample cells 112 where the interrogation of the sample occurs. In this example, the diagnostic unit 104 also includes a sample input port 114 and a cap 116. A sample is provided into the input port 114, and the cap 116 is secured onto the sample input port 114 to enclose the sample in the diagnostic unit 104. The sample is directed into the one or more sample cells 112. In some embodiments, the sample is directed into the sample cells 112 by the action of securing the cap 116. Electrodes 118 arranged in the sample cells are coupled to the reader 102 through the interface 106. The reader 102 operates to interrogate the sample using the electrodes 118 in the sample cells. Examples of the diagnostic unit 104 are illustrated and described in more detail with reference to FIGS. 3-12.

In some embodiments, the diagnostic unit 104 measures at least one characteristic of the sample. Examples of such characteristics include electrical characteristics, such as admittance, conductance, susceptance, and the like. Changes in one or more of characteristics of the sample over time are measured in some embodiments.

In some embodiments, the diagnostic device 100 operates to perform one or more of the following: identify the quantity of a microbe present in a sample, identify the type of microbe present in the sample, and determine whether (and to what extent) microbes present in the sample are sensitive to an antimicrobial. Examples of antimicrobials include an antibiotic, a bactericide, a peptide, a bacteriophage, a chemotherapeutic, or combinations thereof. Examples of microbes include bacteria, fungi, nematodes, cell cultures, and tissues.

In order to determine whether the microbes are sensitive to an antimicrobial, in some embodiments the antimicrobial is included in at least one of the sample cells 112. In some embodiments, multiple sample cells 112 each contain different antimicrobials. In some embodiments, for the purpose of redundancy, which improves the accuracy of the diagnostic result, antimicrobials may exist in multiple sample cells 112. If the microbes present in the sample are sensitive to the antimicrobial, the diagnostic unit 104 will detect changes one or more characteristics of the sample in the corresponding sample cell 112, such as by comparing it to a control cell that contains a sample but no antimicrobial, permitting the diagnostic unit 104 to determine that the microbe is sensitive to the antimicrobial in the sample cell 112.

In some embodiments, identifying a microbe aids in tracking the source of the infection, while microbe counting helps to quantify the severity of the infection, for example.

FIG. 2 is a block diagram of an example reader 102 of the diagnostic device 100. In some embodiments the reader 102 includes a housing 132, electronic components 134, and a diagnostic unit interface 106A. Some embodiments further include a micropump 136. In this example, the electronic components 134 include a power source 142, analog electronics 144 including an AC current source 146 and an AC voltmeter 148, an A/D converter 150, a digital signal processor 160, a central processing unit 162, a computer readable medium 164, a display processor 166, a display device 168, a communication device 170, a heater controller 172 (and heating element), and input device 174. Some embodiments further include a clock and one or more voltage meters. Other embodiments include more or fewer components.

The diagnostic unit interface 106A is a portion of the interface 106, which is configured to couple the reader 102 to the diagnostic unit 104. As one example, the diagnostic unit interface 106A is a card slot configured to receive and electrically connect with the reader interface 106B of the diagnostic unit 104. In this example, an electrical connection is made between the reader 102 and the diagnostic unit to permit the communication of digital or analog electrical signals between the reader 102 and the diagnostic unit 104. Other types of electrical or data communication are used in other embodiments. For example, some embodiments utilize wireless data communication, such as using radio frequency, infrared, or inductive communication devices and signals.

The housing 132 provides a protective enclosure for the reader 102. In some embodiments the housing 132 is formed of plastic. Other embodiments are formed of other materials. The housing 132 includes an interior space which houses at least some of the components of the reader 102, such as the micropump 136 and electronic components 134. In some embodiments, one or more apertures are formed in the housing 132, such as to permit passage of a conduit coupled to the micropump, to permit the diagnostic unit interface 106A to be coupled to the reader interface 106B (FIG. 3), and for a communication port of the communication device 170. In some embodiments, the communication device 170 includes a plurality of communication sub components. For example, in some embodiments the communication device 170 includes an RFID reader, which is used to communicate with the sensor system 192 (and more specifically, with the data storage device 208, all of which are discussed in more detail with reference to FIG. 3). The housing 132 can be formed of one or more materials. In some embodiments, at least a portion of the housing 132 is transparent, such as to permit viewing of the display device 168.

Some embodiments include a micropump system 136. The micropump system 136 is connected through a conduit of the interface 106 (or a separate interface) to the fluidics system 190 of the diagnostic unit 104, and generates a pressure differential to move fluids within the diagnostic unit into the sample cells 112. As discussed with reference to FIG. 3, some embodiments of the diagnostic unit 104 do not include a fluidics system, and accordingly the micropump 136 is not needed in such embodiments. Alternatively, in some embodiments the diagnostic unit 104 itself generates pressure differentials without utilizing the micropump 136.

The power source 142 stores and supplies power to the electronic components 134. In some embodiments the power source 142 is a battery. Some embodiments further include battery charging electronics. In other possible embodiments, the power source 142 includes power supply electronics, configured to receive electrical energy from an external power source, such as mains power, and to convert the electrical energy into a suitable form (such as into a relatively low voltage signal, such as 3, 12, or +/−15V direct current).

The analog electronics 144 are coupled to the diagnostic unit interface 106A. In some embodiments, the analog electronics 144 include an AC current source 146 and an AC voltmeter 148. The AC current source 146 generates AC signals that are provided to a first set of electrodes in the sample cells 112 of the diagnostic unit 104 through the diagnostic unit interface 106A. As one example, the AC current source 146 generates and supplies a continuous AC current. In some embodiments, the AC signal is a sine wave having a frequency in a range from about 100 Hz to about 5 kHz. In some embodiments, the AC signal is a sine wave having a frequency in a range from about 100 Hz to about 10 kHz. Some embodiments have a frequency of about 40 kHz. Some embodiments have a frequency of about 3 kHz.

The AC voltmeter 148 receives analog signals that are generated on a second set of electrodes in the sample cells 112 of the diagnostic unit 104, through the diagnostic unit interface 106A, and determines an AC voltage of the signal. In some embodiments, the AC voltmeter 148 determines a voltage across the second set of electrodes in the sample cells 112, for example. In some embodiments, the AC voltmeter 148 can also be operated to determine a voltage across the first set of electrodes and the AC current source 146.

Some embodiments include a plurality of AC current sources 146 and/or a plurality of AC voltmeters 148 that are directly electrically connected to the electrodes 118, while in other embodiments one or more multiplexers 143 are arranged between the analog electronics 144 and the electrodes 118. The multiplexers 143 and the analog electronics 144 are controlled by the central processing unit 162.

In some embodiments the analog electronics 144 control the period, frequency, voltage, and current optimized to measure and determine the admittance, the conductance, and the constant phase element of the sample in the sample cell 112, and changes in same over time. The analog electronics 144 are controlled by the central processing unit 162 in some embodiments. The A/D converter 150 converts the sensed values to digital values for further analysis by the digital signal processor 160.

The digital signal processor 160 executes algorithms to interpret signals on the electrodes 118, and is controlled by the central processing unit 162. In some embodiments, the digital signal processor 160 accesses a signature database stored in the computer readable storage medium 164, and compares the signals with the signatures. In other embodiments, the digital signal processor 160 directly interprets the signals based on recognition algorithms, or a combination of both. Specific algorithms used by the digital signal processor 160 are determined by the type of sample cell 112 which is determined by the diagnostic unit 104 model number, for example.

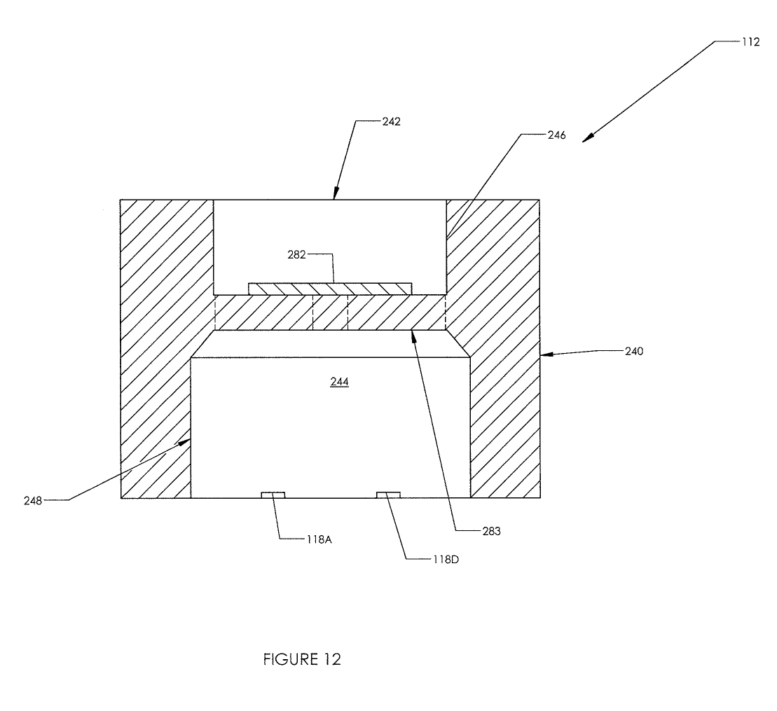

Some embodiments include a central processing unit 162. The central processing unit 162 is an example of a processing device. In some embodiments, the central processing unit 162 controls the overall operation of the diagnostic device 100. For example, the central processing unit 162 controls the operation of the micropump 136 in some embodiments, selects a mode of operation of the electronic components 134, and controls the electronic components 134 according to the selected mode of operation (as discussed in further detail with reference to FIG. 12), and communicates with external devices through the communication device 170.

The computer readable medium 164 is communicatively connected to, or part of, the central processing unit 162, and/or one or more of the other processing devices (e.g., digital signal processor 160 and display processor 166) of the reader 102. An example of a computer readable medium is a computer readable storage device, as discussed herein.

The display processor 166 operates to control the one or more display devices 168 to convey information in a visual form to a user. In some embodiments, the display processor 166 also acts as an input device when the display device is a touch sensitive display. In one example, the display device 168 is a plurality of light sources, such as light emitting diodes (LEDs). As another example, the display device 168 is a two-dimensional display, such as a liquid crystal display (LCD), LED display, and the like.

A communication device 170 is provided in some embodiments to permit communication between the reader 102 and another device, such as a computing device, an RFID storage medium, or a data communication network. In some embodiments, the communication device 170 includes multiple communication devices. In some embodiments the communication device 170 includes a communication port for connection with a communication cable, such as a USB cable or an Ethernet cable. In other embodiments, the communication device is a wireless communication device, such as an RFID reader, a Wi-Fi communication device, a cellular communication device, or a Bluetooth communication device.

Some embodiments further include a heater controller 172 and heating element that is configured to apply heat to the diagnostic unit 104 to achieve and maintain a temperature conducive to microbial growth. In some embodiments, the heating element is arranged in a heating pad (i.e., an incubator warmer), which can be external, or partially external, from the housing of the reader 102, and arranged so that at least a portion of the diagnostic unit abuts the heating pad. In some embodiments, the heating pad is a silicone heating pad. In some embodiments, the heating element is formed of tungsten or nichrome wire. In some embodiments a thermocouple, or other temperature detecting device is provided in the heating pad, in the reader 102, or in the diagnostic unit 104, to provide feedback to the reader 102 to permit the reader 102 to maintain a desired temperature, or range of temperatures, within the sample cells 112. In some embodiments the thermocouple is inserted directly into the electrolytic solution.

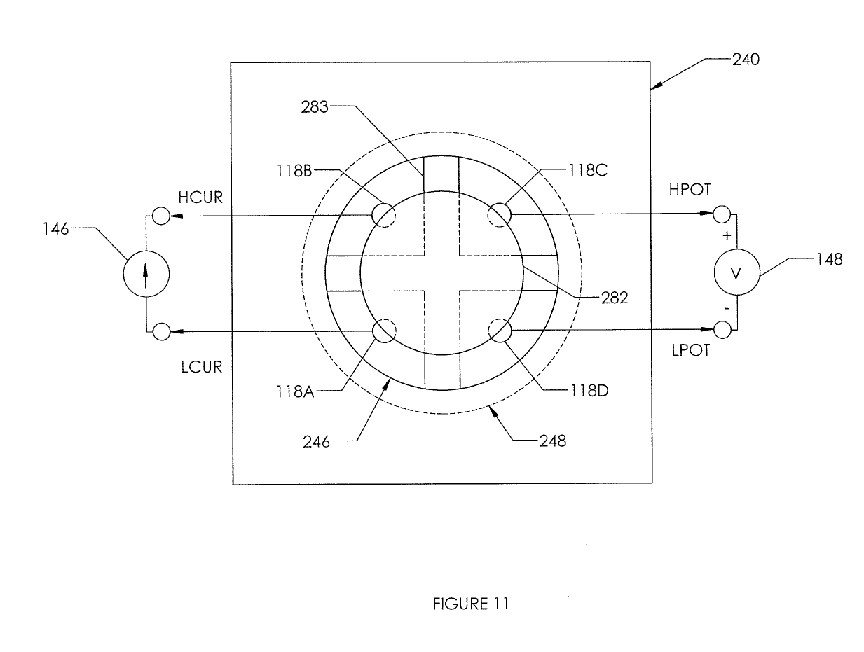

Some embodiments include one or more input devices 174. The input devices 174 can include buttons, switches, touch-sensitive displays, and the like. Other interface devices can also be used, such as an audio (e.g., voice) interface. The input devices can be used to turn the diagnostic device 100 on or off, and to adjust a mode of operation of the device, such as to adjust the device between the first and second states of operation, as described herein with reference to FIG. 11. Other inputs are provided in other embodiments.

The exemplary components of the reader 102 are provided by way of example only. Other embodiments can include more or fewer components. Further, in some implementations, some of the components can be combined into a single component.

In some embodiments, the reader 102 is formed of two or more parts. For example, in some embodiments the reader 102 includes an integral cellular telephone. In another possible embodiment, the reader 102 is configured to receive and cooperate with a cellular telephone. In another embodiment, the reader 102 includes a computing device, such as a mobile computing device (e.g., smart phone, laptop computer, tablet computer, etc.), a desktop computer, or other computing devices. The computing device can be integrated into the reader 102, or external from and in data communication with the reader 102, for example.

Other reader 102 configurations are also possible. For example, another possible configuration of a reader 102 including multiple parts includes a first part and a second part. The first part of the reader includes a housing which contains at least some of the electronic components 134. The second part of the reader has its own housing and forms a warming cradle, including at least the heating element of the heater controller 172. The first part connects with the second part through a first interface, which permits communication between the sensor and the warming cradle. The second part connects with the diagnostic unit 104 through the diagnostic unit interface 106A.

FIG. 3 is a block diagram of an example diagnostic unit 104 of the diagnostic device 100 shown in FIG. 1. In this example, the diagnostic unit 104 includes a housing 188, a fluidics system 190, and a sensor system 192.

Also in this example, the input to the fluidics system 190 includes a sample input port 114 and a cap 116. The example fluidics system 190 includes a filtration system 202, including a fluid source 203, and a sample distribution system 204, including a manifold 206. The example sensor system 192 includes sample cells 112 and electrodes 118.

In some embodiments, the diagnostic unit 104 receives a sample through the sample input port 114. In some embodiments, the diagnostic unit 104 further includes a sample input receptacle having an internal volume suitable to temporarily store part or all of the sample as it is received.

A wide variety of samples can be used in various embodiments. In some embodiments, samples are obtained from a subject suspected of having an infection with a microbe, from a food or water sample, a soil sample, or from a surface or other environmental source. Samples obtained from a subject can include or be obtained from urine, blood, sweat, mucus, saliva, semen, vaginal secretion, vomit, tears, sebum, pleural fluid, peritoneal fluid, gastric juice, earwax, cerebrospinal fluid, breast milk, endolymph, perilymph, aqueous humor, vitreous humor, biomass, mucous membranes, stool sample, infected cells or tissues, lung lavage, cell extracts, biopsies and combinations thereof, for example. Samples can further include sources for yeast, fungi, viruses, nematodes, cell culture, or tissue.

Once the sample has been received into the sample input port 114, in some embodiments a cap 116 is provided, which can be secured onto the sample input port 114. In some embodiments the cap 116 is a locking cap, which includes a locking feature that resists removal of the cap after the cap has been secured to the sample input port 114. In this way, the sample is contained within the housing 188 of the diagnostic unit 104. In some embodiments the housing 188 (including cap 116) forms a sealed enclosure. In some embodiments the sealed housing permits the diagnostic unit 104 to be discarded while continuing to contain the biological materials, which may be considered a biohazard, within the sealed enclosure of the housing 188. Therefore, in some embodiments the diagnostic unit 104 is a single-use disposable unit.

In some embodiments the cap 116 drives a mechanical cam attached to a plunger that creates different pressures within the diagnostic unit 104, which then drives the automation of the fluidics system 190. In other embodiments the automation of the sample handler is driven by a micropump 136 contained in the reader 102.

In some embodiments, the received sample is delivered to the sensor system 192 by the fluidics system 190. In other embodiments, however, the fluidics system 190 is omitted, and manually processed sample that has been re-suspended in the nutrient broth designed to work with the sensor system 192, and may be input by the user directly into the sensor system 192 along with the antimicrobial dispenser 282. Some such embodiments give users the flexibility to customize the device for their specific application. For example, in some embodiments the reader 102 includes a customizing application which permits the users to identify the customizations made to the diagnostic device 100.

In some embodiments, the fluidics system 190 transfers the received sample from the sample input port 114 (or sample input receptacle) to the sensor system 192 after filtering and mixing the sample with nutrient broth from the fluid source 203. In some embodiments the fluidics system 190 is driven by the micropump 136 of the reader 102, shown in FIG. 2. In other embodiments, the filtration system 202 is driven by another source, such as by pressures formed by the insertion and rotation of locking cap 116. In some embodiments, the cap 116 is attached to a cam system wherein turning of the cap causes a plunger to create pressure differentials to drive the fluidics system 190.

In some embodiments, the fluidics system 190 includes a filtration system 202 that filters the received sample. As one example, the filtration system 202 is specialized to handle urine and includes one or multiple filtering stages, such as including a first stage and a second stage. In the case of a urine sample, the first stage of the filtration system 202 may be provided to remove blood and protein from the urine sample. In the second stage, the microbes are removed from the urine sample. The microbes may then be removed from the second stage for further evaluation by the diagnostic unit 104. The remaining urine including wild bacteriophages are passed to a waste receptacle.

In some embodiments, a sample is processed through the filtration system 202 before placing the samples in the sample cells. In some embodiments, for example, the samples are filtered to remove larger particles, cells, and cell debris. In some embodiments, a filtration system 202 is provided that passes the sample through a filter. The filter can have apertures measuring about 5 microns, for example. The filter allows the passage of the microbes while retaining larger particles. The filtration system can further include a secondary filter. The secondary filter can be used to remove unwanted medium in the sample (e.g. urine), such as using a smaller sized filter (e.g., having apertures measuring about 0.45 microns) leaving the bacteria on the surface of the filter so it can be removed and suspended in the nutrient solution for further testing by the diagnostic device 100. In some embodiments, an additional filtering stage is provided between the first and second filtering stages discussed above to capture wild bacteriophage. For example, a filter having 0.22 micron apertures can be used. After a period of time these wild phage can then be reintroduced into the sample cells containing antimicrobials to detect remaining live bacteria since bacteriophage will only attack live bacteria. All bacteria have a host of wild phages in any sample.

Some embodiments of the filtration system 202 also include a fluid source and a mixing device. The fluid source 203 provides a source of a fluid that can be mixed with the sample for use within the sensor system 192. The fluid may be a single fluid or a combination of fluids. An example of a fluid is an electrolytic solution, such as including an electrolyte. One example of an electrolytic solution is a culturing broth. Another example is a culturing broth combined with one or more other culturing broths or other fluids. The electrolyte or nutrient broth should support the growth of the microbe being tested. A sample, as used herein, refers generally to any fluid containing at least a portion of the biological fluid (or any other fluid, material, or other input) received in the sample input port 114, including before or after filtering and/or mixing with another fluid.

To obtain accurate repeatable results, it is desirable that the ionic makeup be tightly controlled within predefined ranges. Further, because real-time monitoring of microbe life signs is desired, the electrolytic solution must support and even stimulate the microbe growth.

The sample distribution system 204 is configured to distribute the received sample to the sample cells 112 in the sensor system 192. In some embodiments, the sample distribution system 204 includes a manifold 206 that evenly mixes and delivers a homogenous sample to at least some of the sample cells 112. Some embodiments include a manifold 206 that does not deliver the sample to all of the sample cells 112, to permit one or more of the sample cells 112 to be used as a control cell. In some embodiments the sample distribution system 204 includes a metering device that provides a substantially equal quantity of the sample to the sample cells 112. In another possible embodiment, fill level sensors on the sample cells 112 operate to provide feedback to the fluidics system 190 to obtain appropriate fill levels of the sample in the sample cells 112.

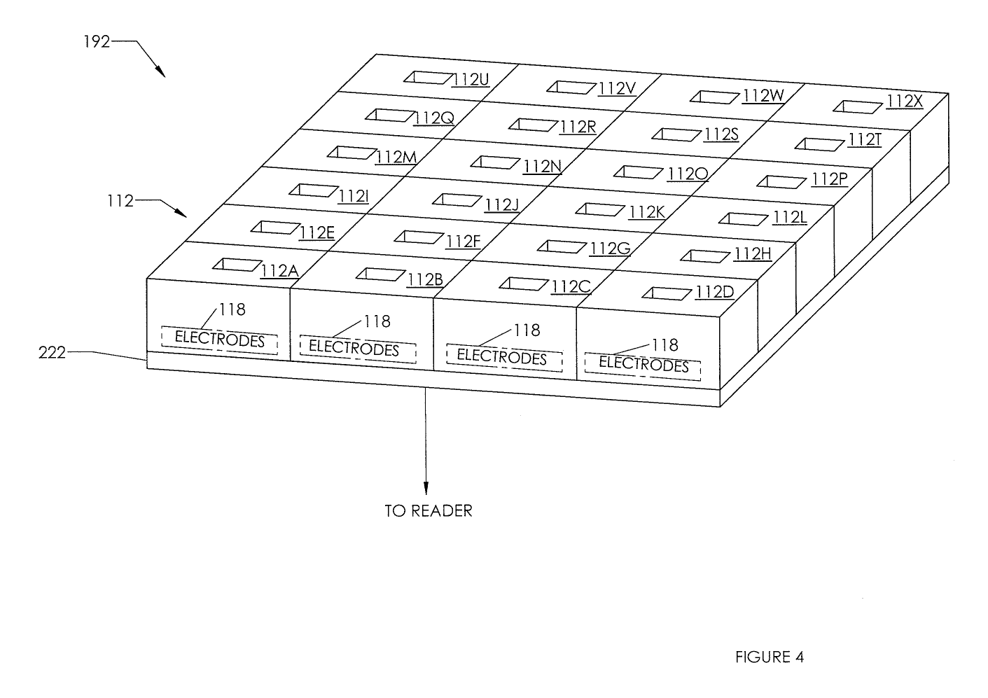

The sensor system 192 includes one or more sample cells 112 and electrodes 118 arranged within the sample cells. An example of the sensor system 192 is illustrated and described in more detail with reference to FIG. 4.

In some embodiments, the sensor system 192 (or elsewhere in the diagnostic unit) includes a data storage device 208 for storing data, such as patient information, diagnostic results, diagnostic unit model number, diagnostic unit serial number, or combinations thereof. In some embodiments, the data storage device 208 is a passive read-write RFID device, for example. In some embodiments, the data storage device 208 can be written to and read by an RFID reader of the communication device 170 (FIG. 2) of the reader 102.

A reader interface 106B is provided in some embodiments to permit electrical or data communication between the diagnostic unit 104 and the reader 102 (FIG. 2). As one example, the reader interface 106B includes a card-type interface having a plurality of electrical contact pins that is insertable into a corresponding card slot of the diagnostic unit interface 106A of the reader 102. Other interfaces are used in other embodiments, such as a data communication port (e.g., USB, serial, etc.) or a wireless communication device. The reader interface 106B permits communication between the reader 102 and the electrodes 118 for interrogation of the sample in the sample cells 112.

In some embodiments, the diagnostic unit 104 is coupled to the reader 102 (FIG. 2) to conduct a diagnostic evaluation for a period of time of at least 1 minute or less, 5 minutes or less, 10 minutes or less, 15 minutes or less, 20 minutes or less, 30 minutes or less, 45 minutes or less, or 60 minutes or less.

FIG. 4 is a schematic perspective view of an example sensor system 192 of the diagnostic unit shown in FIG. 3. In this example, the sensor system 192 includes a base substrate 222, a plurality of sample cells 112 (including sample cells 112A to 112X), and electrodes 118.

In this example, the sensor system 192 includes a base substrate 222. An example of a base substrate 222 is a circuit board, such as a printed circuit board. Another example of the base substrate 222 is a flexible substrate, such as a flex circuit. The base substrate can be formed of one or more layers and includes at least one insulating layer. One or more conductive layers are provided in some embodiments, such as a ground plane or one or more layers including electrical traces. In some embodiments, the base substrate 222 includes electrical conductors between the electrodes and the reader interface 106B (not visible in FIG. 4).

The sample cells 112 are arranged on and supported by the base substrate 222. In some embodiments the sample cells 112 are formed of a single piece of material, while in other embodiments the sample cells 112 are individual pieces. In yet other embodiments, a subset of the sample cells 112 are formed of a single piece (e.g., each row of sample cells 112 can be formed of a single piece of material). In one example embodiment, the sample cells 112 are made of plastic, such as molded plastic or injection molded plastic. The sample cell 112 material can be constructed out of a medically approved insulating material. It is preferred that the material does not have an adverse effect on the growth of microbes in the sample. The sample cells 112 are coupled to the base substrate 222 by a fastener, such as adhesive or other bonding layer or material. Further, some embodiments include one or more materials between the sample cells 112 and the base substrate 222, such as a gasket layer. In some embodiments, the sample cells 112 are bonded to the base substrate 222. The one or more fasteners that connect the sample cells to the base substrate 222 are preferably configured to inhibit leakage of the sample or other fluid out of and between the sample cells 112. In some embodiments, the sample cells 112 are molded around electrodes in a lead frame.

In some embodiments, the sensor system 192 includes a plurality of sample cells 112. In the illustrated embodiment, the sensor system 192 includes an arrangement of 24 sample cells 112A-X. In some embodiments the sample cells are arranged in a grid of rows and columns. In this example, the sample cells are arranged in four rows and six columns. In other embodiments, the sensor system 192 includes a plurality of sample cells 112 in a range from 2 to 50, or 2 to 48, or 2 to 36, or 2 to 24. The sample cells can be arranged in one or more rows (e.g., 1, 2, 3, 4, 5, 6, 7, 8, or more rows). The sample cells can have cubed, cylindrical, or rectangular shapes, for example, and can also have other configurations, such as hexagonal shapes, etc. Further, in some embodiments the sample cells 112 are all formed of a single piece of material. For example, the sample cells 112 are formed of a single piece of plastic material that is molded around a lead frame which forms the electrodes, in some embodiments.

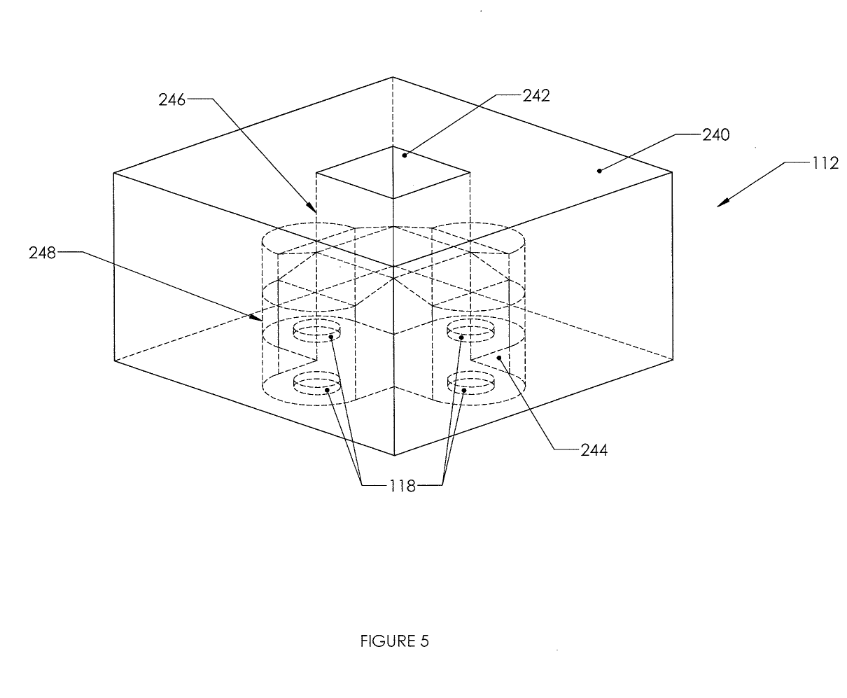

The sample cells 112 include therein a sample chamber. Electrodes 118 are arranged within the sample cells 112, to interact with the sample for determining the one or more characteristics of the sample, as discussed in further detail herein. More specific examples of the sample cell 112 are illustrated and described in more detail with reference to FIGS. 5-10 herein.

In some embodiments, the shape of the sample chamber is tuned to provide more accurate measurements of the characteristics, including positioning the electrodes such that the shapes of the electric fields that are generated by the electrical signals applied to the electrodes are optimized for signal-to-noise performance.

In some embodiments, the sample cells 112 are manufactured so that the size and shape of the sample chambers are substantially the same. In this way, the sample cells have similar dimensional constants, permitting the diagnostic device to make comparisons between measured characteristics of one or more sample cells as compared with the measured characteristics of one or more other cells, as discussed in further detail below.

In some embodiments, the electrodes 118 are formed on the base substrate 222. To improve the seal between the sample cells 112 and the base substrate 222, the electrodes can be formed within recessed regions formed in the surface of the base substrate 222, such that the surfaces of the electrodes are flush with the surface of the base substrate 222. In some embodiments the recessed regions are nanowells. In another possible embodiment, the electrodes are formed on the surface of the substrate 222. In yet other possible embodiments, the electrodes can be arranged in other locations, such as on the walls of the sample chamber of the sample cells 112. In a further embodiment, the sample cells 112 include a bottom surface, and the electrodes 118 are arranged on the interior side of the bottom surface, or side walls near the bottom of the sample cells 112.

The electrodes 118 can be made from one or more of a variety of materials, such as any noble metal, a metal coated with graphene or graphenol-like substances, or combinations of one or more of these (e.g., metal alloys). As one example, the electrodes 118 are formed of metal pins. In another possible embodiment, the electrodes 118 are gold plated. In some embodiments, the electrodes are gold plated electrodes patterned onto the base substrate 222. In some embodiments, the electrodes 118 are coated with a graphenol-like substance applied directly to copper traces on the base substrate 222 before or after the solder-mask is applied (in the case of a printed circuit board, for example). Other embodiments use gold or graphene directly printed onto a flexible plastic substrate to make flex circuits. Other embodiments utilize electrodes formed from the exposed tips of a lead frame molded into the plastic diagnostic unit 104.

In some embodiments, the size of each of the electrodes and the distance apart of each of the electrodes is precisely controlled. In embodiments, the size of the electrodes is substantially the same, that is, the electrodes have a difference in size of less than 5%, 1%, 0.1%, 0.01%, or 0.001%. In some embodiments, the distance between the electrodes in different sample cells is substantially the same, that is, having a difference in distance between electrodes of less than 5%, 1%, 0.1%, 0.01%, or 0.001%. In some embodiments, the electrode sizes and distances between electrodes are controlled to a difference of 1% of less.

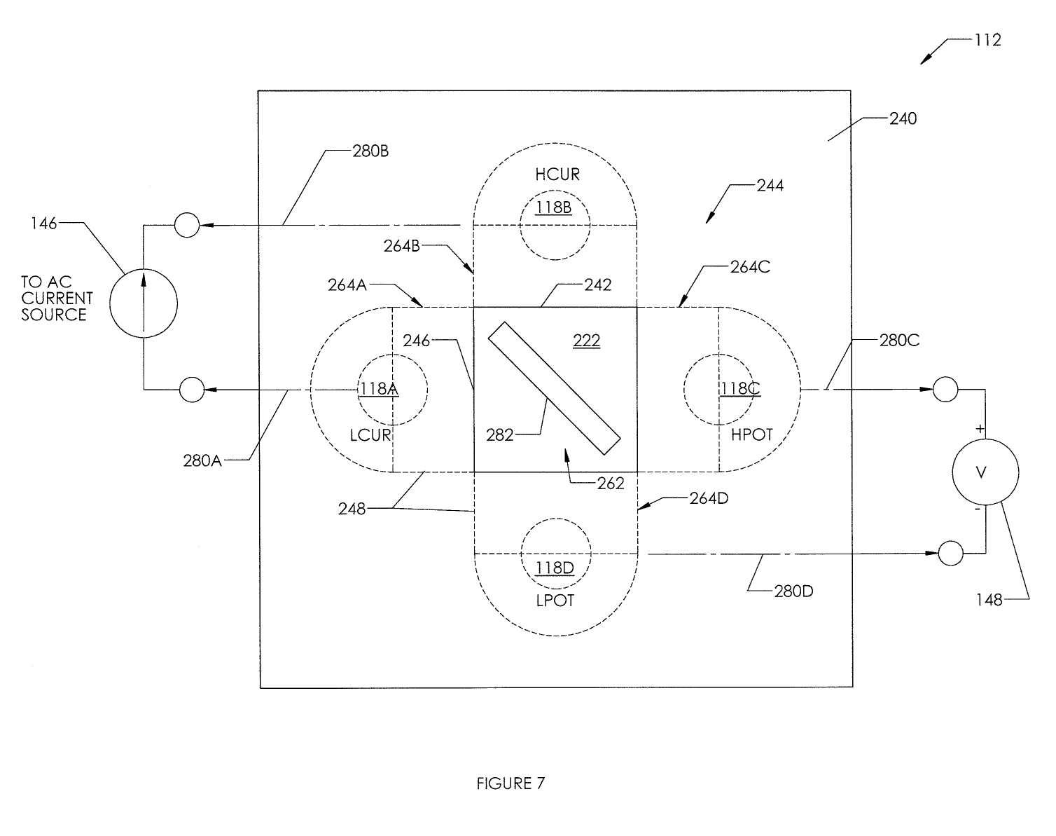

In some embodiments, the sample cells further include antimicrobial dispensers 282, as illustrated and described in more detail with reference to FIGS. 7 and 12-13.

FIGS. 5-7 illustrate an example of a sample cell 112.

FIG. 5 is a schematic perspective view of the example sample cell 112. Portions of the sample cell 112 are depicted as transparent to illustrate the interior structure of the sample cell 112. In this example, the sample cell 112 includes a body 240, an input opening 242, a sample chamber 244, and electrodes 118. In some embodiments the sample chamber 244 includes a chimney 246 and an interrogation region 248.

A sample is received through the opening 242. In some embodiments, the input opening 242 is coupled to the manifold 206 of the sample distribution system 204 shown in FIG. 3. The opening 242 includes a coupling port in some embodiments, such as configured to be connected to a fluid delivery conduit, such as tubing, to connect the opening 242 with the manifold 206. The sample distribution system 204 delivers the sample through the manifold and into the input opening 242. In another possible embodiment, the sample is provided directly by a user or another device into the input opening 242, such as using a pipette or other sample container or fluid delivery conduit.

After the sample has passed through the input opening 242, the sample then passed through the chimney 246 of the sample chamber 244, and then into the interrogation region 248.

Electrodes 118 arranged within the interrogation region 248 of the sample chamber 244 are electrically coupled to electronic circuitry, such as the analog electronics 144 of the reader 102 (shown in FIG. 2), which operate to generate electrical signals at one or more of the electrodes 118, and to detect electrical signals at one or more of the other electrodes 118. The detected electrical signals are then used to evaluate one or more characteristics of the sample.

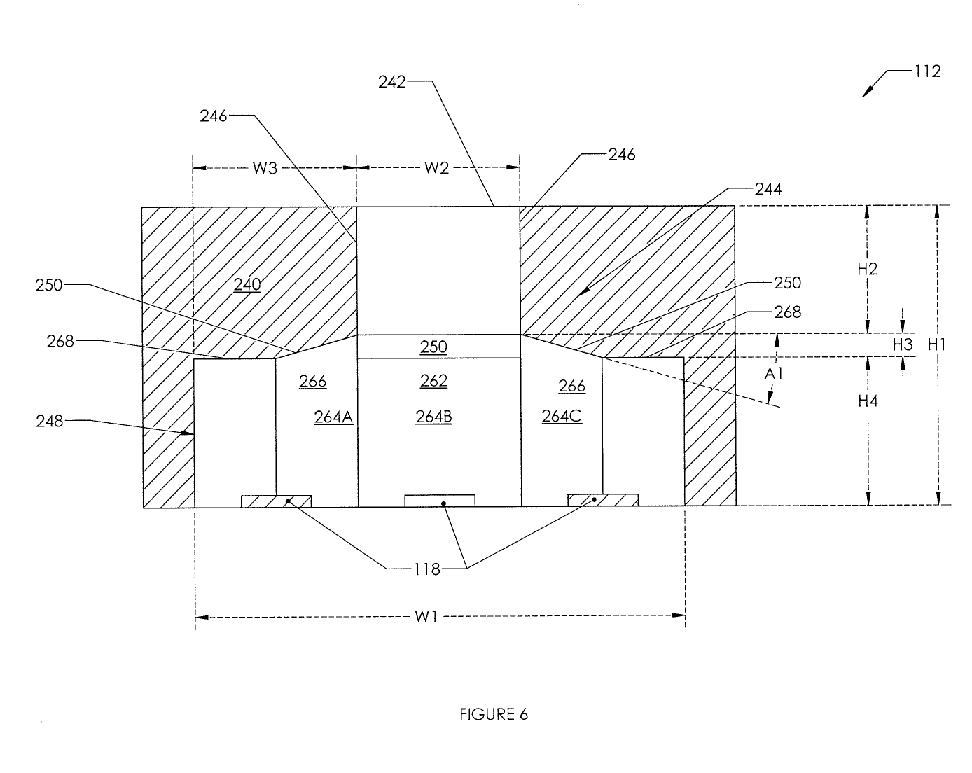

FIG. 6 is a schematic side elevational view of the example sample cell 112, shown in FIG. 5. In this example, the sample cell 112 includes the body 240, the input opening 242, the sample chamber 244, and electrodes 118.

In this example, the sample chamber 244 includes both the interrogation region 248 and the chimney 246. The interrogation region 248 is sized to hold a precise volume of the sample. It is preferred that the interrogation region be entirely filled before interrogating the sample, to provide uniform results among the sample cells 112. When electrical signals are applied to one or more of the electrodes 118, electrical currents as well as electric fields are generated within the sample. If the interrogation region is not entirely filled, the currents and electric fields produced within the sample are modified, potentially resulting in a change in the one or more measured characteristics of the sample. Therefore, for a given type of sample (e.g., blood, urine, etc.), the volume of the interrogation region 248 is selected to be small enough that it can be filled by the sample based on sample volumes that can typically be obtained for that given type of sample. In some embodiments, the volume of the interrogation region 248 is in a range from about 0.1 mL to about 10 mL, or from about 0.5 mL to about 2 mL, or from about 1 mL to about 1.5 mL.

The chimney 246 extends from the interrogation region and is provided in some embodiments to contain an additional volume of the sample, in addition to the volume of the interrogation region 248. In this way, the volume of the sample does not have to be precisely measured to match the volume of the interrogation region 248 exactly, but rather can be somewhat greater than the volume of the interrogation region 248—up to the combined volume of the interrogation region 248 and the volume of the chimney 246. In some embodiments the volume of the chimney is in a range from about 0.01 mL to about 2 mL, or from about 0.1 mL to about 0.2 mL, or about 0.14 mL. In some embodiments the volume of the chimney is in a range from about 5% to about 20% of the volume of the interrogation region, or from about 1% to about 10%, or about 10%.

The configuration of the chimney 246 permits some variation in the sample volume without significantly affecting the measured characteristics of the sample. For example, the chimney 246 has a cross-sectional dimension (W2) that is much less than the cross-sectional dimensions (W1) of the interrogation region 248. Additionally, the chimney 246 extends away from the interrogation region 248, and does not provide a return path for current to flow through the chimney 246. As a result, when an electrical signal is applied to one or more of the electrodes 118, very little electrical current is conducted through any portion of the sample that is within the chimney. Therefore, whether the level of the sample is at or near the top of the chimney 246 (i.e., at opening 242), at or near the bottom of the chimney 246, or somewhere in between, the one or more measured characteristics of the sample are not significantly changed. The chimney 246 therefore provides a sample volume buffer that permits variations in the volume of the sample up to the total volume of the chimney 246.

In this example, the chimney has a width (W2) and an equal depth (D2, not shown), and a height (H2). The volume of the chimney is W2×D2×H2. The volume can therefore by adjusted by increasing or decreasing any of these dimensions. For example, the volume can be increased or decreased by adjusting the height (H2) of the chimney. In one example, the width W2 is in a range from about 1 mm to about 20 mm, or from about 2 mm to about 6 mm, or from about 4 mm to about 5 mm, or about 4.5 mm. In this example, the height H2 is in a range from about 1 mm to about 50 mm, or from about 5 mm to about 10 mm, or from about 6 mm to about 8 mm, or about 7 mm.

In some embodiments, the interrogation region 248 includes a central region 262 and radially extending arms 264 (including arms 264A to 264D). As best shown in FIG. 7, in some embodiments the interrogation region 248 has a cross-sectional shape of a plus, a cross, or an “X”.

In this example, the central region 262 has a square horizontal cross section and a rectangular vertical cross section. For example, the central region 262 has a width (W2), an equal depth (D2) (not shown in FIG. 6), and a height (H3+H4). In some embodiments, the width (W2) is the same as the width (W2) of the chimney. In other embodiments, the width of the central region 262 is different than the width of the chimney. In one example, the height (H3+H4) of the central region 262 is in a range from about 2 mm to about 35 mm, or from about 5 mm to about 20 mm, or from about 10 mm to about 14 mm, or about 12 mm.

Four arms 264 extend radially from the central region 262. Each of the arms has a straight region 266 and terminates in a semi-cylindrical shaped region 268. The straight region 266 has a tapered height that varies from H4 to (H3+H4). The semi-cylindrical shaped regions 268 have a diameter equal to the depth (D3, not shown) of the straight region 266, and a height (H4). In one example, the length of the straight region 266 is in a range from about 1 mm to about 20 mm, or from about 2 mm to about 6 mm, or from about 4 mm to about 5 mm, or about 4.5 mm. The length can be greater than or less than the length W2 of the central region 262. In this example, the diameter of the semi-cylindrical shaped regions 268 are in a range from about 1 mm to about 20 mm, or from about 2 mm to about 6 mm, or from about 4 mm to about 5 mm, or about 4.5 mm. The height (H4) of the semi-cylindrical region is in a range from about 2 mm to about 30 mm, or from about 5 mm to about 20 mm, or from about 8 mm to about 12 mm, or about 9.6 mm.

In some embodiments, an upper portion 250 of the interrogation region 248 has a tapered shape. If bubbles are present in the sample within the interrogation region 248, such bubbles could potentially change the one or more measured characteristics of the sample. The tapered shape of the upper portion 250 collects the bubbles as they rise to the top of the interrogation region 248 and directs the bubbles toward and into the chimney 246. The bubbles then rise through the chimney 246 to the surface of the sample and exit the sample. The accuracy of the sample measurements are therefore improved. In this example, the upper portion 250 has a taper angle A1. In some embodiments the taper angle A1 is in a range from about 10 degrees to about 80 degrees, or from about 10 degrees to about 45 degrees, or from about 10 degrees to about 20 degrees. Some embodiments have an angle A1 of about 15 degrees. Although this example illustrates the tapered upper portions 250 terminating before the semi-cylindrical shaped regions 268, in other possible embodiments the tapered upper portion 250 extends to the ends of the arms 264.

The exemplary dimensions described herein are provided by way of example only. Other embodiments can have dimensions that are greater or less than the dimensions discussed herein. Additionally, the overall dimensions of the sample cell 112 can be any desired dimensions greater than (or equal to) the dimensions of the sample chamber 244.

FIG. 7 is a schematic top plan of the example sample cell 112, shown in FIG. 5. FIG. 7 also illustrates the base substrate 222, electrical conductors 280 (including conductors 280A-D), and an example antimicrobial dispenser 282, as well as the AC current source 146 and AC voltmeter 148 of the reader 102 (shown in FIG. 2).

As previously described, this example of the sample cell 112 includes a body 240 and a sample chamber 244. The sample chamber 244 includes an opening 242, a chimney 246, and an interrogation region 248. The interrogation region 248 includes a central region 262 and arms 264 (including arms 264A-D). The sample cell 112 is arranged on a base substrate 222 in some embodiments, and electrodes 118 (including electrodes 118A-D) and the antimicrobial dispenser 282 are arranged thereon.

The cross-sectional shape of the example sample chamber 244 is shown in FIG. 7, which has the general shape of a plus, cross, or “X”, in which the arms 264 extend radially from the central region 262 and extend at right angles to adjacent arms. For example, arms 264A and 264C extend perpendicular to arms 264B and 264D, while arm 264A extends parallel with arm 264C, and arm 264B extends parallel with arm 264D.

In this example, electrodes 118 are arranged on the base substrate 222 at one end of each of the arms 264. Each of the electrodes 118A-D is electrically coupled to an electrical conductor 280A-D, respectively. The electrical conductors 280 are coupled to the analog electronics 144 of the reader 102. For example, electrodes 118A and 118B are electrically coupled to the AC current source 146, and electrodes 118C and 118D are electrically coupled to the AC voltmeter 148. Electrode 118A operates as a low current (L_{CUR}) terminal. Electrode 118B operates as a high current (H_{CUR}) terminal. Electrode 118C operates as a high potential (H_{POT}) terminal. Electrode 118D operates as a low potential (L_{POT}) terminal. In some embodiments, additional electrical connections are possible, such as by using a multiplexer, as discussed herein. In some embodiments, the AC voltmeter 148 is capable of reading voltages across electrodes 118A and 118B, as well as across electrodes 118C and 118D, or other combinations of the electrodes.

In some embodiments, the measurement of conductance is insensitive to the capacitive reactance at the driven or forced electrodes (i.e., 118A-B). The measurement is also insensitive to capacitive reactance at the voltage sensing electrodes (i.e., 118C-D) because the reactance is insignificant compared to the input impedance of the voltage sensing instrument at frequencies at or above a few hundred Hz. Low frequency performance is improved by the use of larger electrodes 118 to produce more capacitance from the electrode polarization.

In some embodiments, the four electrode sample cell (including any one of the examples shown in FIGS. 5-12) provides direct measurement of conductivity scaled by a geometry constant. Scaling the conductivity of the sample cell 112 contents by the geometry constant yields the measured conductance.

Geometry constant (ξ) as defined herein relates conductance (G) and conductivity (κ) as:

$$ξ ≡ \frac{{G}_{0}}{{κ}_{0}}$$

where G₀ and κₒ are reference values at a particular temperature.

The geometry constant can be computed from the conductance at a temperature G(T) with knowledge of the temperature coefficient (ζ) as:

$$ξ = \frac{G ( T )}{{κ}_{o} ( 1 + ζ ( T - {T}_{0} ) )} .$$

An average of a group of individual geometry constants is thus:

$$\overline{ξ} = \frac{{ξ}_{1} + {ξ}_{2} + … + {ξ}_{N}}{N} .$$

If the group of cells contain a variety of electrolytic contents, the average geometry constant can be indicated as an effective value (ξ̅e) under the assumption that the conductivities and temperature coefficients are reasonably matched. Then, a unique scaling constant can be derived for each conductance curve as a function of time, so that the scaled curves appear as they would if the cells had well matched geometry constants near the average of the actual geometry-constant values. A set of scaling constants can be obtained from the above geometry constants as:

$${C}_{1} = \frac{{\overline{ξ}}_{e}}{{ξ}_{1}} , {C}_{2} = \frac{{\overline{ξ}}_{e}}{{ξ}_{2}} , … , {C}_{N} = \frac{{\overline{ξ}}_{e}}{{ξ}_{N}} .$$

Under the above assumptions, κ₀ cancels out of the scaling constants. For example:

$${C}_{1} = \frac{\overline{ξ}}{{ξ}_{1}} = \frac{1 + \frac{{G}_{2} ( {T}_{0} )}{{G}_{1} ( {T}_{0} )} + … + \frac{{G}_{N} ( {T}_{0} )}{{G}_{1} ( {T}_{0} )}}{N} \vert {G}_{n} ( {T}_{0} ) = \frac{{G}_{n} ( T )}{1 + ζ ( {T}_{Cell n} - {T}_{0} )} .$$

These equations applied over an entire set of data produces a Cₙ set for each point in time.

The inability to measure temperature accurately in the presence of thermal gradients, such as during a rapid warm-up period, confounds the ability to compute accurate Gₙ(T₀) values and hence the above scaling constants. However, averaging the scaling constant of each cell over a period of time following the warm-up yields a single, useful value. Each conductance at temperature Gₙ(T) function of time can be multiplied by the corresponding scaling constant (Cₙ) to get the scaled function:

G_{ξ}(T)ₙ=CₙGₙ(T).

For the case with all the test cells at the same temperature, the scaling constant equation from the above example simplifies to.

$${C}_{1} = \frac{1 + \frac{{G}_{2}}{{G}_{1}} + … + \frac{{G}_{N}}{{G}_{1}}}{N} .$$

Accurate conductivity measurements of electrolytic solutions facilitate quantitative comparisons of the ionic content within multiple sample cells 112. If an electrolytic solution includes a nutrient media (sometimes alternatively referred to herein as broth) with the addition of living microbes, the presence of the microbe is detectable as an effective increase in ion content due to metabolic activity of the microbe on the broth components. The increase in conductivity caused by the presence of live bacteria can be expressed as the difference in conductivity measurements obtained with a sample cell containing broth with bacteria (Cell 1) and a sample cell containing broth only (Cell 0). In this example, the sample cells have four electrodes each (118A-D), one pair (118A-B) to deliver electrical current (“forced electrodes”) from the AC current source 146, and one pair (118C-D) to sense the voltage developed inside the sample cell in response to the current (“sensed electrodes”), as measured by the AC voltmeter 148. If two sample cells are dimensionally well matched, a scaled difference in conductivity is obtained from the difference in conductance measurements as:

$$ξ \cdot {κ}_{B} = {G}_{1} - {G}_{0} = \frac{{If}_{1}}{{Vs}_{1}} - \frac{{If}_{0}}{{Vs}_{0}}$$

where κ_{E} is the proportion of the conductivity in Cell 1 attributable to bacterial activity, G₁ is the conductance of Cell 1, G₀ is the conductance of Cell 0, ξ₁ and ξ₀ are the respective dimensional constants of Cell 1 and Cell 0, If₁ and If₀ are the respective currents flowing in Cell 1 and Cell 0, and Vs₁ and Vs₀ are the respective sensed voltages of Cell 1 and Cell 0. If the cells are not well matched, the respective values ξ₁ and ξ₀ are applied to Cell 1 and Cell 0:

$${κ}_{B} = \frac{{G}_{1}}{{ξ}_{1}} - \frac{{G}_{0}}{{ξ}_{0}} = {κ}_{1} - {κ}_{0}$$

where κ₁ and κ₀ are the respective conductivities of the electrolytic solutions in Cell 1 and Cell 0.

Since the magnitude of κ_{B} increases monotonically with the number of bacteria colony forming units (CFU) present in sample cell 1, it provides a basis for counting (or quantifying) bacterial concentration and real-time monitoring its change across time.

By the same method used to obtain κ_{B}, another sample cell containing broth, bacteria and an antimicrobial agent (Cell 3) has a portion of the conductivity (κₖ) attributable to bacterial activity countered by the antimicrobial agent provided by the antimicrobial dispenser 282:

$${κ}_{A} = \frac{{G}_{B}}{{ξ}_{3}} - \frac{{G}_{0}}{{ξ}_{0}} = {κ}_{3} - {κ}_{0}$$

Relationships between κ₀, κ_{B}, and κ_{A} can be analyzed in real time to detect increasing and decreasing bacterial metabolic activity. From these data relationships, the effectiveness of the given antimicrobial agent can be evaluated.

In some embodiments, temperature compensation is beneficial when quantifying conductance measurements of microbial broth solutions, or when comparing results from separate tests. Some broth recipes yield conductivities with linear coefficients of temperature typically around 20,000 ppm/° C. Rapid test results require mixing and transferring broth cultures into sample cells and not waiting for thermal equilibrium conditions before beginning the test. Microbial broth cultures are incubated to a normal human body temperature, such as 35° C., 37° C., or in a range from about 35° C. to 37° C. (normal human body temperature) to promote growth, so some control of the temperature is necessary, but fast and highly accurate feedback control of the incubator temperature would add cost and complexity to the measurement system. Compensation can be applied to test data if the temperature of the broth is monitored during testing, but accurate direct monitoring may add cost and complexity particularly undesirable for a single-use disposable sensor. An indirect method of temperature compensation that does not require control or monitoring of the temperature would be advantageous in cost and performance. The following describes such a method appropriate when testing a plurality of sample cells in unison that is used in some embodiments.

A control cell containing only broth (Cell 0 above) can be used to indicate temperature if the conductivity at a reference temperature and the temperature coefficient are known:

$$T = {T}_{0} + \frac{{κ}_{0} ( T ) - {κ}_{0} ( {T}_{0} )}{ζ}$$

where T is the broth temperature at which the conductivity κ₀(T) is measured, T₀ is the reference temperature at which the broth conductivity κ₀(T₀) is known and ζ is the temperature coefficient.

If another cell containing broth with bacteria (Cell 1 above) is measured at the same temperature T as Cell 0, the conductivity at the reference temperature can then be expressed as:

κ₁(T₀)=κ₁(T)−ζ(T−T₀)

Combining these last two equations gives:

κ₁(T₀)=κ₁(T)−κ₀(T)+κ₀(T₀)

Assumptions using this method are: the microbial concentration added to the broth has negligible effect on the temperature coefficient, and the sample cells differ in temperature by a negligible amount. Notice that no measurement of temperature is necessary during the test, and that the temperature coefficient need not be known.

An alternative method can be used to express the conductance of a sample cell, or the conductivity of its contents, at an arbitrary temperature value within the temperature range occurring over the test duration. Using again Cell 0 and Cell 1 as defined above and with the same assumptions, the measured broth conductance in Cell 0 at an arbitrary test point can be defined as G_{OA}. A set of correction ratios (Rn) can then be generated at each test point as:

Rₙ=G_{0A}/G₀ₙ

Then, if each measured conductance value, G₀ₙ, is multiplied by the appropriate value of Rₙ all G₀ₙ conductivity values will be corrected to the G₀ₙ value.

This same set of correction ratios, Rₙ, applied to the conductance of Cell 1 will result in removing the temperature dependence of the broth from the broth plus bacteria conductance values, for example.

Temperature Compensated G₁ₙ≡G_{1comp}=Rₙ*G₁ₙ

Remaining differences between the broth only and broth plus bacteria conductance will then be due entirely to the presence of the bacteria.

Note that G_{OA} need not be an actual data-point measurement. In a preferred embodiment G_{OA} may be defined as the mean average value of the measured G₀ₙ data set. Then, G_{1comp} represents the data as though the temperature had been held constant at the average value attained over the test duration.

Bacteria, when attacked by bacteriophages, admit quantities of ions which can be detected by this measurement technique even against the background conductivity of the medium. Bacteriophages can be cultivated so that they will attack one and only one bacteria species or sub-species. Additionally, the bacteriophages can be selected so that it causes the bacteria to release ions during the initial attack. When using a bacteriophage that attacks one and only one bacteria, identification of the bacteria is possible by observing the ionic surge and the eventual reduction in live bacteria which occurs during the period (i.e., the first five to fifteen minutes) after the introduction of the cultivated bacteriophage and the target bacteria enabling rapid identification of the bacteria.

Ultimately, antimicrobials—specifically antibiotics and bacteriophages—cause the metabolism to slow down or cease, yielding one or more detection mechanisms specific to a given antimicrobial agent's ability to eradicate the microbe. Monitoring the microbes during a period (i.e., the first hour) after the antimicrobial attack gives a good indication of the final outcome of the antimicrobial.

Algorithms to identify bacteria include monitoring the electrical properties and thermal properties while the bacteria are in the presence of antimicrobials including bacteriophage and in some embodiment's simultaneously testing in separate test cells the bacteria's reaction to antibiotics. Combined analysis across sample cells give increased accuracy. Furthermore, adding redundant identification test cells increase accuracy of the identification.

Algorithms to identify bacteria sensitivity to antibiotics can also include monitoring electrical and thermal properties taking into account results from bacterial identification tests and using redundancy to statistically improve the accuracy of the antimicrobial sensitivity test results.

In this embodiment, antimicrobials (including antibiotics or bacteriophages) are impregnated into a fibrous substrate designed to store a premeasured concentration of the antimicrobial in a moisture free environment and further designed to eluent the antimicrobial into the nutrient solution when the two come into contact with each other.

An experimental setup was performed. The performance of the experimental setup was tested using a work process that counted the bacteria using standard plating techniques. Bacteria were counted (by making culture plates of three bracketing dilutions of the bacteria used in the experiment or the source bacteria) before the test began. Source antimicrobials were tested against source bacteria using overnight culturing techniques. Post experiment each cell was plated to show that the broth was/wasn't contaminated during the experiment; the antimicrobial did/didn't work against the bacteria; and the exact growth of the control bacterial (by making culture plates from two bracketing dilutions made from the contents of the bacteria-only cell). In general, each experiment used four cells: broth only, microbe only, antimicrobial to generate a true positive, antimicrobial to generate a true negative.

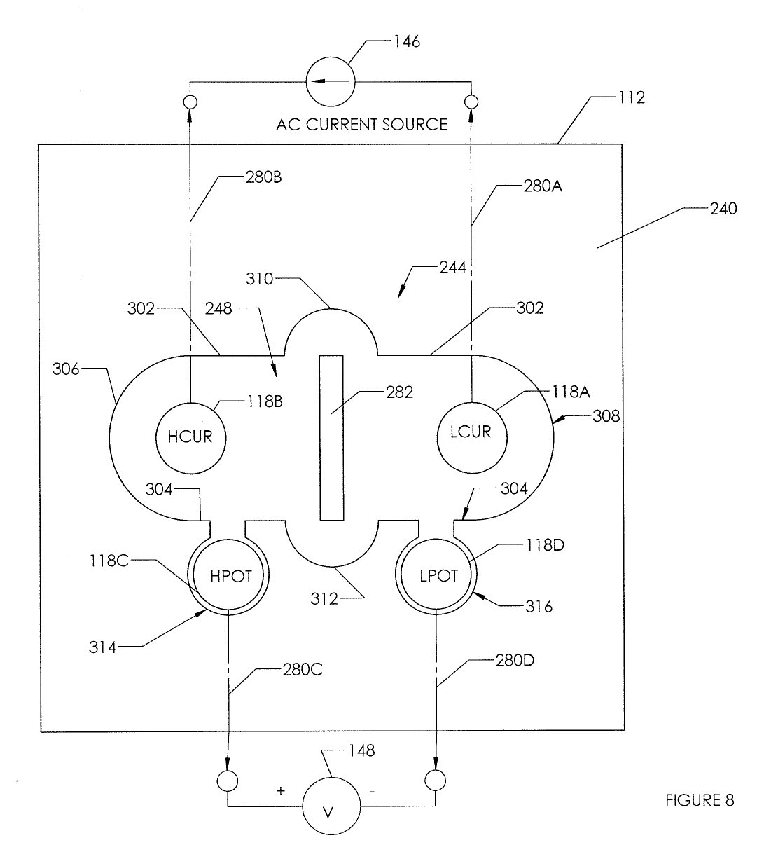

FIG. 8 is a cross-sectional top view of another example sample cell 112 and another example electrode configuration. The sample cell 112 includes a body 240 having a sample chamber 244 formed therein, including an interrogation region 248. Electrodes 118A-D are arranged in the sample chamber 244, as well as the antimicrobial dispenser 282. Electrical conductors 280A-D provide electrical connections to the electrodes 118A-D, to couple the electrodes 118A-D to the analog electronics 144 of the reader 102.

The electrodes 118 include electrode 118A, which operates as the low current (L_{CUR}) terminal, and is coupled to the AC current source 146 through the electrical conductor 280A. The electrode 118B operates as the high current (H_{CUR}) terminal, and is coupled to the AC current source 146 through the electrical conductor 280B. The electrode 118C operates as the high potential (H_{POT}) terminal, and is coupled to the AC voltmeter 148 through the electrical conductor 280C. The electrode 118D operates as the low potential (L_{POT}) terminal, and is coupled to the AC voltmeter 148 through the electrical conductor 280D.

The sample chamber 244 includes an interrogation region 248 where interrogation of the sample occurs. In this example, the interrogation region 248 has an elongated shape including longitudinal sidewalls 302 and 304 and semi-circular ends 306 and 308. In some embodiments, the elongated interrogation region 248 includes recesses 310 and 312 formed at the sidewalls 302 and 304, which provide additional space in the interrogation region 248 for the antimicrobial dispenser 282.

The driven or forced electrodes 118A and 118B are arranged within the elongated interrogation region, which when energized by the AC current source 146, generate an AC current that flows through the elongated interrogation region from the high current electrode 118B to the low current electrode 118A.

The sample chamber 244 also includes sensing regions 314 and 316. The sensing regions 314 and 316 both extend from a common sidewall 304 of the elongated interrogation region 248. The sensing regions 314 include narrowed arm portions that extend perpendicular to the sidewall 304 and terminate in a larger circular region at the ends of the narrowed arm portions. The sensed electrodes 118C and 118D are arranged in the larger circular regions of the sensing regions 314 and 316, respectively. In some embodiments, the sample chamber 244 is symmetrical about a central axis extending between the recesses 310 and 312.

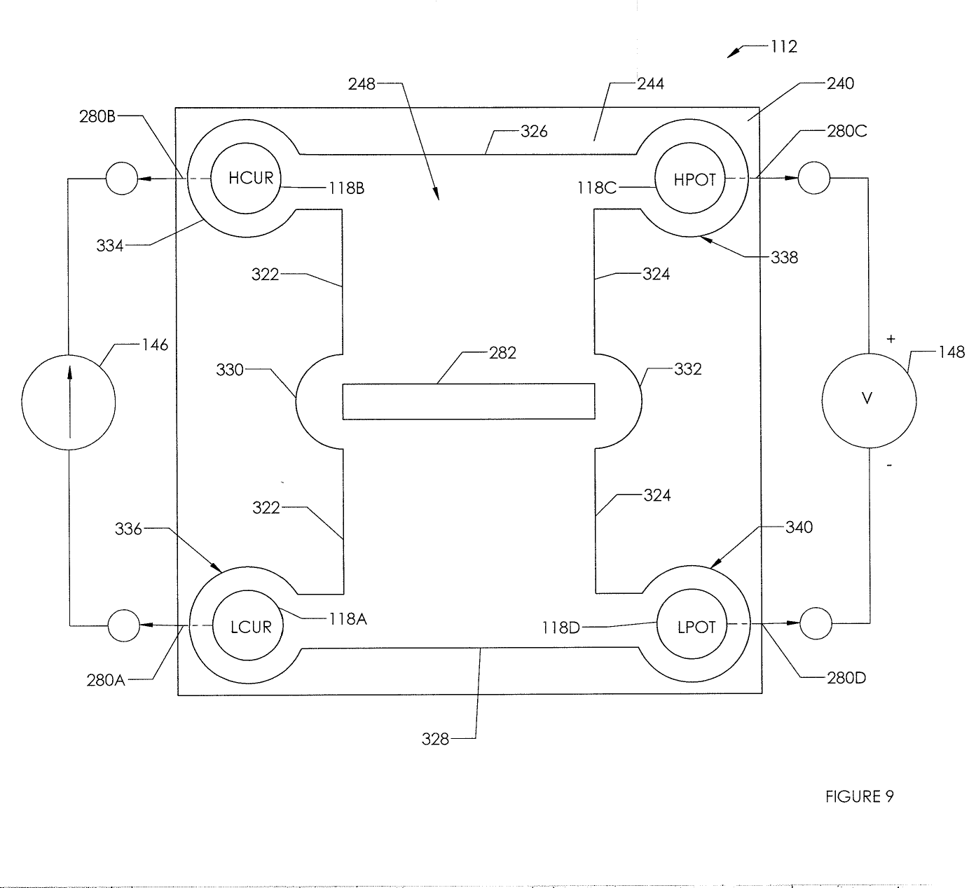

FIG. 9 is a top cross-sectional view of another example sample cell 112 and another example electrode configuration. The sample cell 112 includes body 240 and sample chamber 244, including an interrogation region 248.

The interrogation region 248 includes a straight elongated region having longitudinal sidewalls 322 and 324 and flat end walls 326 and 328. Recesses 330 and 332 are formed at the sidewalls 322 and 324 in some embodiments, adjacent the location of the antimicrobial dispenser 282.

Forced regions 334 and 336 extend from opposite ends of the sidewall 322 in a common direction. The forced regions 334 and 336 include narrowed arm portions that extend from the sidewall 322. A portion of each of the narrowed portions of the forced regions 334 and 336 shares a common wall with the flat end walls 326 and 328, respectively. The forced regions 334 and 336 terminate in larger circular end regions.

The forced electrodes 118B and 118A are arranged in the larger circular end regions of the forced regions 334 and 336. The high current electrode 118B is arranged in the forced region 334 and the low current electrode 118A is arranged in the forced region 336, for example. The high current electrode 118B and the low current electrode 118A are coupled to the AC current source 146 through electrical conductors 280B and 280A, respectively.

Sensed regions 338 and 340 similarly extend from opposite ends of the sidewall 324 in a common direction, parallel to but opposite the direction of the forced regions 334 and 336. The sensed regions 338 and 340 include narrowed arm portions that extend from the sidewall 324. A portion of each of the narrowed portions of the sensed regions 338 and 340 shares a common wall with the flat end walls 326 and 328, respectively. The sensed regions 338 and 340 terminate in larger circular end regions.

The sensed electrodes 118C and 118D are arranged in the larger circular end regions of the sensed regions 338 and 340. The high potential electrode 118C is arranged in the sensed region 338 and the low potential electrode 118D is arranged in the sensed region 340, for example. The high potential electrode 118C and the low potential electrode 118D are coupled to the AC voltmeter 148 through electrical conductors 280C and 280D, respectively.

In this example, the interrogation region 248 is symmetrical about central axes extending through the end walls 326 and 328, and extending through the recesses 330 and 332. Accordingly, the functions of the electrodes can be swapped accordingly without modifying the operation of the sample cell 112.

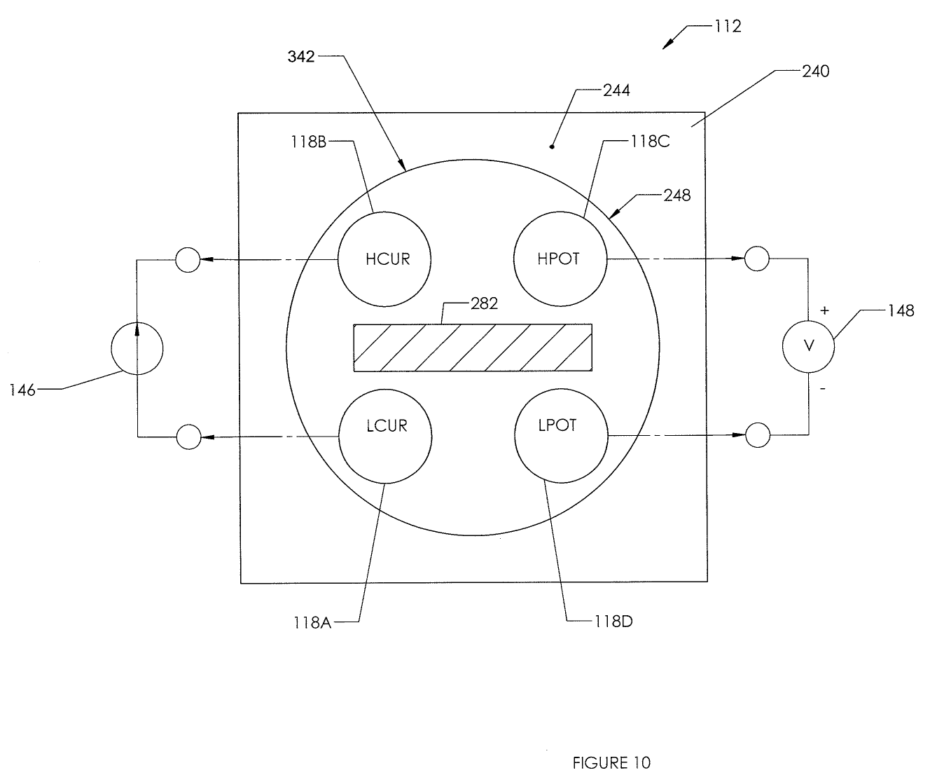

FIG. 10 is a cross-sectional top view of another example sample cell 112 and another example electrode configuration. The sample cell 112 includes a sample chamber 244 having an interrogation region 248.

In this example, the interrogation region 248 has a cylindrical shape having a single sidewall 342. All of the electrodes 118A-118D are arranged at the bottom of the sample cell 112 within the cylindrical interrogation region 248. The antimicrobial dispenser 282 is also arranged within the sample cell 112.

FIGS. 11 and 12 illustrate another example sample cell 112 and another example electrode configuration. FIG. 11 is a cross-sectional top view and FIG. 12 is a cross-sectional side view. This example sample cell 112 includes a sample chamber 244 (FIG. 12) having an interrogation region 248.

In this example, the interrogation region 248 has a cylindrical shape having a single sidewall. All of the electrodes 118A-118D are arranged at the bottom of the sample cell 112 within the cylindrical interrogation region 248. A chimney 246 having a cylindrical shape extends from input opening 242 to interrogation region 248. The antimicrobial dispenser 282 is arranged in a horizontal position on top of an antimicrobial dispenser support 284, which extends across the chimney 246, having the shape of a cross or X-shape.

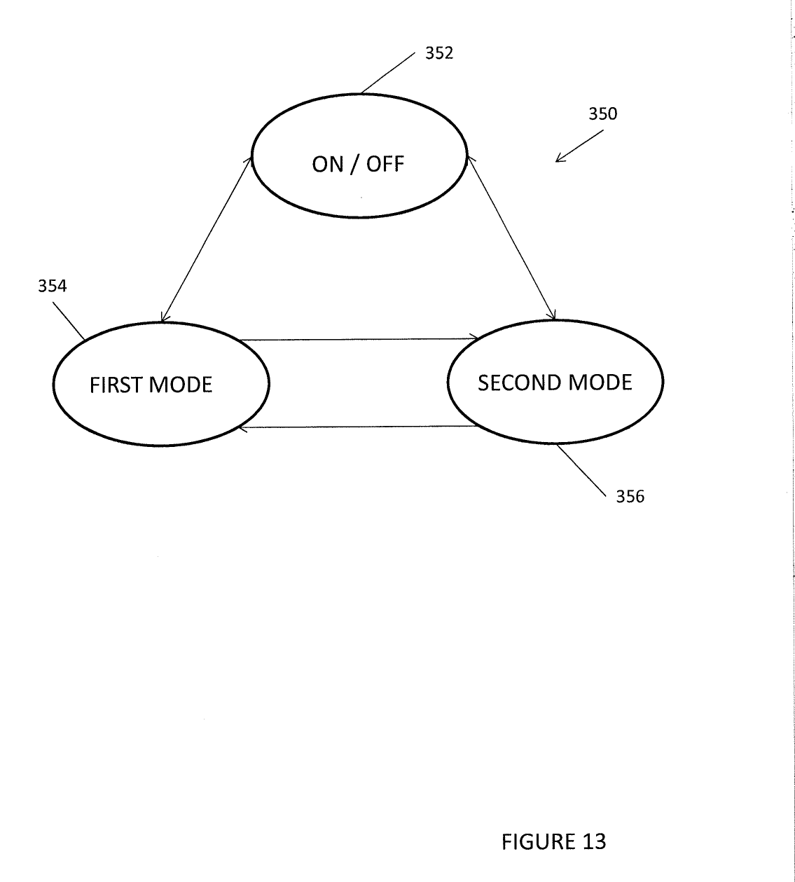

FIG. 13 is a state diagram 350 illustrating an example of the operation of the diagnostic device 100, and also illustrates a method of operating a diagnostic device 100. In this example, the diagnostic device 100 operates in states 352, 354, and 356.

The diagnostic device 100 begins at state 352 when the diagnostic device 100 is turned on. Prior to being turned on, the diagnostic device 100 is prepared for interrogating a sample, such as by adding a suitable quantity of the sample into the sample input port 114 (FIG. 3) of the diagnostic unit 104. Once turned on, the diagnostic device 100 transitions to one of the states 354 or 356.

When the diagnostic device operates in the state 352, the diagnostic device utilizes all four electrodes 118 to perform measurements on the sample. In some embodiments, a first set of the forced electrodes (e.g., 118B and 118A) are energized by the AC current source 146 to generate a current flow through the sample. The second set of sensed electrodes (e.g., 118C and 118D) are then used by the AC voltmeter 148 to detect one or more characteristics of the sample.

For example, in some embodiments the diagnostic device 100 operates to measure conductance of the sample. The conductance measurement is then used to count the quantity of bacteria present in the sample. In some embodiments the quantity of bacteria are determined as a quantity of colony forming units (CFU).

As another example, in some embodiments the diagnostic device 100 operates to identify a type of bacteria present in the sample. To identify the type of bacteria, the diagnostic device 100 monitors the conductance of the sample over time across a plurality of the sample cells. At least some of the sample cells include antimicrobial dispensers including different antimicrobials. For example, identification of the bacteria is possible by observing the ionic surge (and corresponding increase in conductance) and the eventual reduction in live bacteria (and corresponding reduction in conductance) which occurs during the period (i.e., the first five to fifteen minutes) after the introduction of an antimicrobial that is effective at attacking the bacteria present in the sample cell 112. The diagnostic device 100 can therefore monitor for the changes in conductance that occur in sample cells having an effective antimicrobial, and can similarly determine that little to no change in conductance occurs in other sample cells that do not have an effective antimicrobial. Additionally, the conductance can be compared to one or more other control cells, such as a control cell containing a control fluid absent any of the sample, and/or a control cell containing a control fluid and the sample but no antimicrobial.

When the diagnostic device operates in the second mode 356, two or more of the electrodes in one or more of the sample cells 112 to measure one or more characteristics of the sample. For example, in some embodiments, diagnostic device 100 operates to measure the admittance of the sample. Admittance can be computed, for example, as the forced current divided by the voltage between the forced electrodes.

In some embodiments, the second mode 356 utilizes only two of the electrodes 118 within a sample cell. Alternatively, the electrodes can be operated in pairs to utilize four electrodes for the admittance measurement.

In some embodiments, the electrodes are controlled using the electronic components of the reader 102 (shown in FIG. 2).

The second state 356 can be used to take impedimetric measurements of the sample to monitor chemical processes and biological activity. In particular, in some embodiments the reader 102 includes electronic components including impedimetric-based electronics for detection and real-time monitoring and quantification of bacteria in nutrient solution (broth culture). The impedimetric electronics rely on an admittance change within a microbial culture, resulting from a change in ionic content, produced by metabolization of compounds by microorganisms within the culture media. Impedimetric measurements offer advantages of convenience and rapid results over other techniques, such as plating techniques for microbial colony counts.

The admittance measurement can be made with an AC signal. In general, the admittance of the sample cell 112 is complex. A susceptive component of the admittance is apparent due to a capacitance that is generated from a charge double-layer, also known as electrode polarization, which forms at each electrode-sample interface. The bulk conductivity of the electrolytic solution contained within the particular geometry of a sample cell 112 determines the conductive component of the admittance. Changes in the concentration and or type of ions in the electrolytic solution produce changes in both the susceptive and conductive components. For admittance/impedance modeling, the cell can be represented as a capacitor and resistor connected in series. More sophisticated models, such as those that apply a constant phase element to include distributed effects, are beneficial for a detailed analysis.

The efficaciousness of the constant phase element in modeling the cell admittance as a function of frequency may be indicative of a distribution of relaxation times or ionization energies within a cell, resulting in random electrical noise that varies inversely as a power of frequency. The constant phase element also provides insight to variability attributed to electrode surface roughness that can confound cell-to-cell repeatability. The charge double-layer that embodies the cell capacitance, and varies according to ion concentration and ion type and temperature, can also respond to problematic influences such as precipitates, biofilms, or bubbles at the electrodes.

Furthermore, Van der Waals forces at the electrode surfaces support film growth that diminishes the capacitive response to changes in ion concentration within the electrolytic solution. This results in an additional change in the admittance, and a reduction in responsivity, as a function of time during a measurement sequence. The random variables, noise and film growth, limit the signal-to-noise ratio available for measurement of microbial colony forming units (CFU) per unit volume of electrolytic solution.

To avoid much of the limitations imposed by measuring predominantly the capacitance at low frequency, measurements can be performed at higher frequency so that the capacitive susceptance contributes less to the total admittance. The conductance of the bulk electrolyte then dominates the measured cell admittance. However, the distributed nature of the cell admittance confounds applying the lumped resistor-capacitor (series RC) model that would allow sufficient isolation of these parameters by merely changing frequency. Indeed, this measured conductance is observed to vary in relation with the capacitance. Also, for a given fractional change in ion concentration, observations show the resulting fractional change in capacitance is typically greater than the fractional change in conductance by more than one order of magnitude. So the “conductance” parameter exhibits less noise compared to the “capacitance” parameter, but also develops less response to changes in ion concentration.

Therefore, while the second state 356 can be used to measure admittance and evaluate microbe sensitivity, the first state 354 can be used to obtain extended accuracy and dynamic range beyond what is available through the second state 356.

In some embodiments, application of the four-terminal techniques used during the first state 354 involve two terminals supplying an electrical current through a test sample, and two terminals with which the subsequent voltage drop therein is sensed. Embodiments of the four-terminal cell operating during state 354 are less sensitive to effects at the forced/driven electrodes than when operating in the second state 356 by excluding effects of electrode polarization instead of merely excluding series impedance of terminals and interconnects. Additionally, the first state can also provide a direct measurement of the electrolytic solution conductivity scaled by a geometry dependent sample cell factor.

The diagnostic device 100 can use the admittance measurements, for example, to determine antibiotic sensitivity of the microbial present in the sample. For example, the diagnostic device 100 can determine that the microbial has a low, moderate, or high sensitivity to the antimicrobial present in the sample cell.

Some embodiments include one or more additional states, not shown in FIG. 11. For example, some embodiments include a fluid processing state, during which the fluidics system 190 is activated to process and distribute the received sample into the sample cells 112.

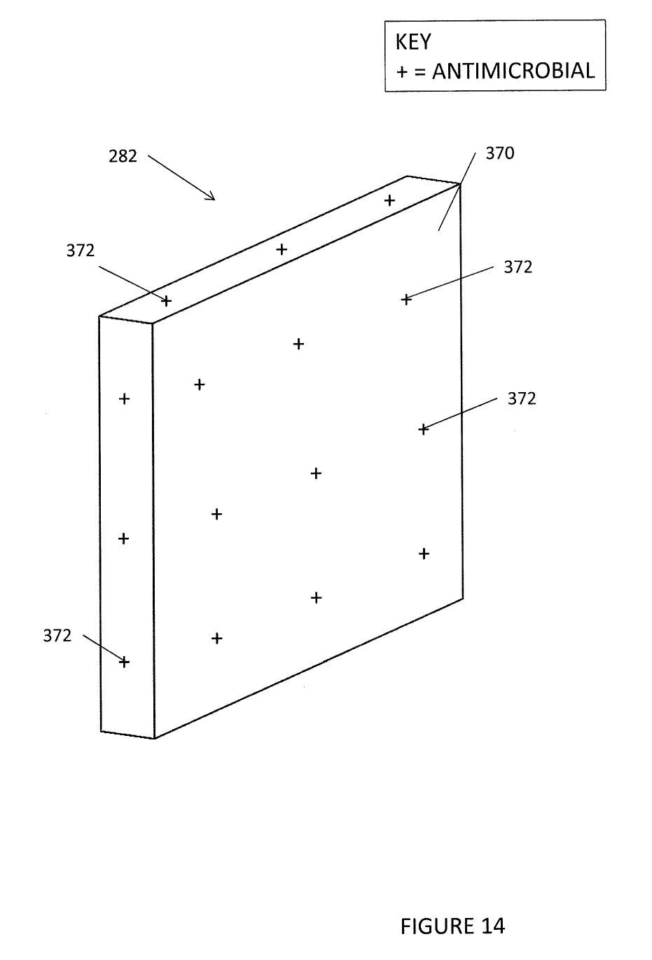

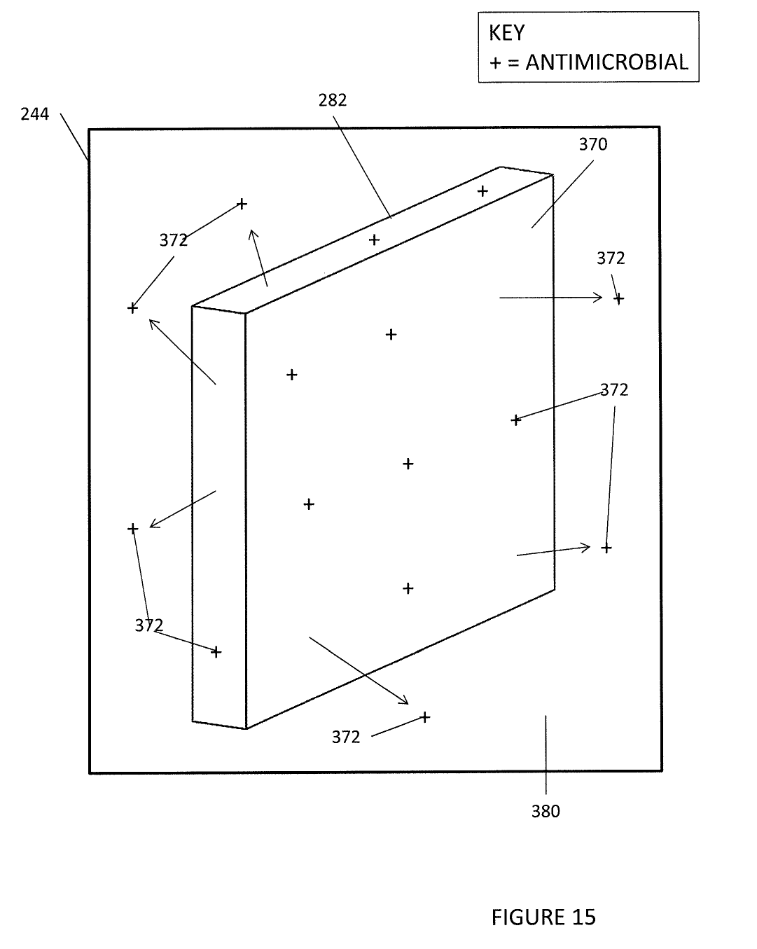

FIGS. 14-15 illustrate an example of an antimicrobial dispenser 282.

FIG. 14 is a perspective view of the example antimicrobial dispenser 282. In this example, the antimicrobial dispenser 282 includes a carrier material 370 and an antimicrobial 372.

The carrier material 370 is a piece of material such as paper, cloth, and the like. In some embodiments the carrier material 370 includes a fastener configured for attaching the carrier material 370 inside of a sample cell 112 (e.g., to the interior of the sample cell 112 itself, or to the base substrate 222, such as shown in FIG. 4).

In some embodiments the carrier material 370 is a thin sheet of material, having a thickness that is much less than (e.g., <10% of, or <1% of) its length and width. This provides increased surface area for interaction with the sample.

The antimicrobial 372 is carried by the carrier material 370. In some embodiments, the antimicrobial 372 is applied to the outside of the carrier material 370. In some embodiments, the antimicrobial 372 is also contained within the carrier material 370. The antimicrobial dispenser (including the antimicrobial 372 and carrier material 370) are dry prior to use.

This antimicrobial dispenser 282 can have various possible shapes in different embodiments, including rectangular, circular, cylindrical, square, triangular, or other shapes. In some embodiments, the face surfaces of the carrier material 370 of the antimicrobial dispenser 282 are slightly hardened against moisture and the edges give access to a fibrous material that easily wicks moisture thus forcing premeasured antimicrobials 372 to eluent into the surrounding liquid. One embodiment of the antimicrobial dispenser 282 uses a specific bacteriophage or specifically design bacteriophage cocktail as the antimicrobial.

One example of an antimicrobial dispenser is the SENSI-DISC™ susceptibility test discs available from Becton, Dickinson and Company, of Franklin Lakes, N.J., containing an antibiotic drug.

In other possible embodiments, the antimicrobial dispenser 282 includes a bacteriophage or bacteriophage cocktail. The bacteriophage is a virus that infects and replicates within bacteria. For example, for detection of urinary tract infections, bacteriophage are selected that are specific for that set of bacteria which can include E. coli, Staphylococcus aureaus, Klebsiella, Proteus, Pseudomonas, and Enterobacter. Examples of bacteriophages that can be used include phages T1, T4 57, VD13, 92, PB-1, or other specially cultivated bacteriophage of interest used alone or in combination. A concentration of bacteriophages can be identified by plaque forming units (PFU) per milliliter.

In some embodiments the bacteriophage have one or more, or all, of the following features: is cultivated and isolated so it attacks one and only one species; inserts DNA through a hole in the bacteria's cell wall so potassium ions are rapidly released; has a long shelf life when lyophilized (e.g., dried shelf life of 2 years or more); revives rapidly when rehydrated; if targets a sub-species, co-exists in a cocktail targeting the species; will attack bacteria regardless of initial bacterial concentration and whether bacteria is in exponential growth phase; has a rapid life cycle (e.g., less than 30 minutes to lysis or shorter); and is a comprehensive blend of phage to minimize any resistant bacteria masking an effective attack—has enough different phage targeting multiple subspecies of a single species to eliminate virtually all bacteria in a sample. These features can be found in Caudovirales phages, for example.

FIG. 15 is a perspective view of the example antimicrobial dispenser 282 shown in FIG. 14 arranged inside of a sample chamber 244 of a sample cell 112. The sample chamber 244 includes a sample 380. FIG. 15 also illustrates the dispensing of the antimicrobial 372 into the sample 380.

In this example, the antimicrobial dispenser 282 is arranged within the sample chamber 244 of a sample cell 112. In some embodiments, the antimicrobial dispenser 282 is fastened inside of the sample chamber 244 in a vertical orientation, as shown. The vertical orientation increases the surface area of the carrier material 370 that is exposed within the sample chamber 244. In other possible embodiments, the antimicrobial dispenser 282 is fastened horizontally. In some embodiments, the antimicrobial dispenser 282 is positioned within the chimney 246 (FIG. 5 and FIGS. 11-12) of the sample cell.

When the sample 380 is provided into the sample chamber 244 or chimney 246, the sample wets the antimicrobial dispenser 282. When wetted, the antimicrobial 372 is released from the carrier material and is disbursed into the sample 380. Due to the large surface area and relatively small internal volume, a large proportion of the antimicrobial 372 is quickly dispensed into the sample 380.

In some embodiments, a plurality of sample cells 112 include different antimicrobial dispensers 282 containing different antimicrobials. For example, in some embodiments at least 10 sample cells 112 each contain an antimicrobial dispenser 282 for dispensing a different antimicrobial. As one example, the antimicrobials are at least 10 different bacteriophages, that lyses a certain species of bacteria. The diagnostic device 100 can then operate to monitor the 10 sample cells to determine whether the microbial present in the sample cell is affected by the antimicrobial, and if so, the identity of the microbial can be determined, for example. In some embodiments, at least one or more of the sample cells 112 include an antimicrobial dispenser 282 that dispenses an antibiotic. Typically at least two sample cells 112 serve as controls, in which case the sample cells 112 may not include an antimicrobial dispenser 282, or alternatively may include an antimicrobial dispenser 282 carrier material 370 without an antimicrobial 372. In some embodiments, a first control sample cell contains an electrolyte solution and does not contain the sample 380 (and microbes contained therein) or an antimicrobial 372. A second control sample cell contains an electrolyte solution and the sample (and microbes contained therein), but does not include an antimicrobial 372. Additional control sample cells are present in some embodiments, such as containing an electrolyte and antimicrobial dispenser including a bacteriophage or an electrolyte and an antimicrobial dispenser including an antimicrobial other than a bacteriophage. Many different embodiments with differing numbers of sample cells to perform different antimicrobial sensitivity tests, microbial identification tests or microbial counting are possible.

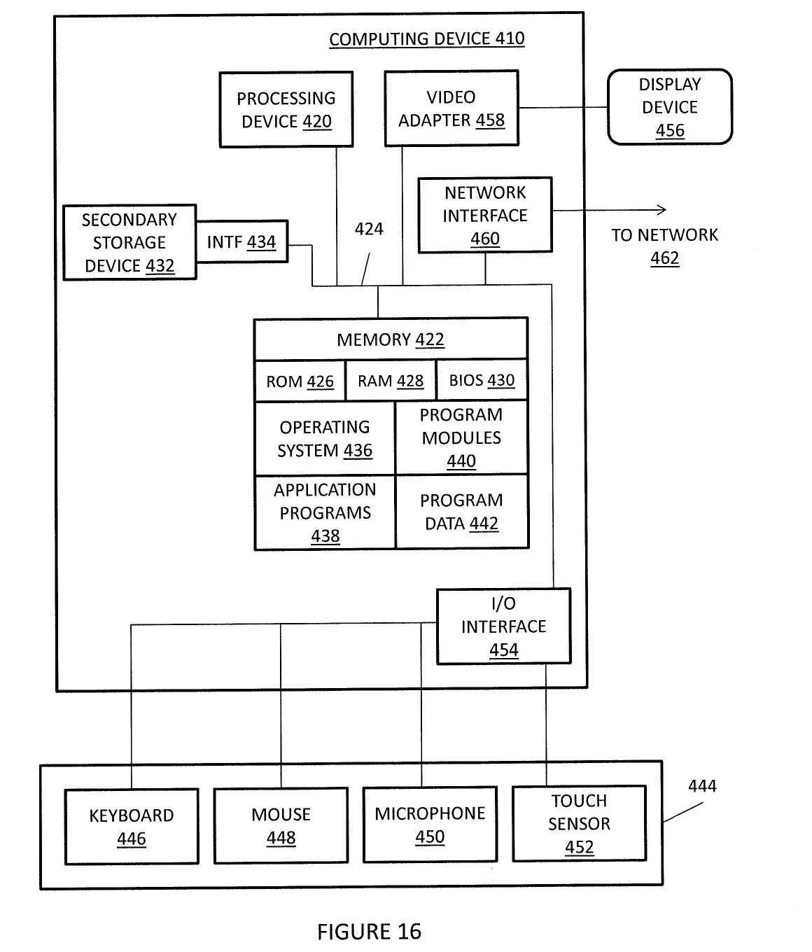

FIG. 16 is a schematic block diagram illustrating an exemplary architecture of a computing device 410 that can be used to implement aspects of the present disclosure. For example, the computing device 410 can be coupled to the diagnostic device 100 through the communication device 170 of the reader 102 (FIG. 2). In another possible embodiment, the computing device 410 is part of the reader 102 (such as to provide the CPU 162, computer readable medium 164, display processor 166, display device 168, communication device 170, and power source 142). By way of example, the computing device will be described below as a separate computing device 410.

The computing device 410 includes, in some embodiments, at least one processing device 420, such as a central processing unit (CPU). A variety of processing devices are available from a variety of manufacturers, for example, Intel or Advanced Micro Devices. In this example, the computing device 410 also includes a system memory 422, and a system bus 424 that couples various system components including the system memory 422 to the processing device 420. The system bus 424 is one of any number of types of bus structures including a memory bus, or memory controller; a peripheral bus; and a local bus using any of a variety of bus architectures.

Examples of computing devices suitable for the computing device 410 include a desktop computer, a laptop computer, a tablet computer, a mobile computing device (such as a smart phone, an iPod® or iPad® mobile digital device, or other mobile devices), or other devices configured to process digital instructions.

The system memory 422 includes read only memory 426 and random access memory 428. A basic input/output system 430 containing the basic routines that act to transfer information within computing device 410, such as during start up, is typically stored in the read only memory 426.

The computing device 410 also includes a secondary storage device 432 in some embodiments, such as a hard disk drive, for storing digital data. The secondary storage device 432 is connected to the system bus 424 by a secondary storage interface 434. The secondary storage devices 432 and their associated computer readable media provide nonvolatile storage of computer readable instructions (including application programs and program modules), data structures, and other data for the computing device 410.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media. Additionally, such computer readable storage media can include local storage or cloud-based storage.

A number of program modules can be stored in secondary storage device 432 or memory 422, including an operating system 436, one or more application programs 438, other program modules 440 (such as the software engines described herein), and program data 442. The computing device 410 can utilize any suitable operating system, such as Microsoft Windows™, Google Chrome™, Apple OS, Google Droid™, Google Ice Cream™, and any other operating system suitable for a computing device.

In some embodiments, a user provides inputs to the computing device 410 through one or more input devices 444. Examples of input devices 444 include a keyboard 446, mouse 448, microphone 450, and touch sensor 452 (such as a touchpad or touch sensitive display). Other embodiments include other input devices 444. The input devices are often connected to the processing device 420 through an input/output interface 454 that is coupled to the system bus 424. These input devices 444 can be connected by any number of input/output interfaces, such as a parallel port, serial port, game port, or a universal serial bus. Wireless communication between input devices and the interface 454 is possible as well, and includes infrared, BLUETOOTH® wireless technology, 802.11a/b/g/n, cellular, or other radio frequency communication systems in some possible embodiments.

In this example embodiment, a display device 456, such as a monitor, liquid crystal display device, projector, or touch sensitive display device, is also connected to the system bus 424 via an interface, such as a video adapter 458. In addition to the display device 456, the computing device 410 can include various other peripheral devices (not shown), such as speakers or a printer.

When used in a local area networking environment, a wide area networking environment (such as the Internet), or a personal area network, the computing device 410 is typically connected to the network 462 through a network interface 460, such as an Ethernet interface or wirelessly, such as using any one or more of the wireless communication devices noted above. The network interface 460 can interface with many different kinds of networks, in some embodiments. Other possible embodiments use other communication devices. For example, some embodiments of the computing device 410 include a modem for communicating across the network 462 (such as the internet or a cellular network, for example).

For example, in some embodiments an application program 438 operates to transfer patient information for storage in the data storage medium 208 (FIG. 3), and similarly to receive diagnostic results from the diagnostic device 100 and transfer such results across the network to another computing device.

In some embodiments, the computing device 410 transfers diagnostic results from the diagnostic device 100 to the network for storage in a cloud data storage system. Similarly, the computing device 410 operates in some embodiments to transfer digital data to the cloud data storage device and for further analytic processing, such as when the analytic processing required is too intensive for the computing device 410 or the diagnostic device 100.

The computing device 410 typically includes at least some form of computer readable media. Computer readable media includes any available media that can be accessed by the computing device 410. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 410. Computer readable storage media does not include computer readable communication media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 16 is also an example of programmable electronics, which may include one or more such computing devices, and when multiple computing devices are included, such computing devices can be coupled together with a suitable data communication network so as to collectively perform the various functions, methods, or operations disclosed herein.

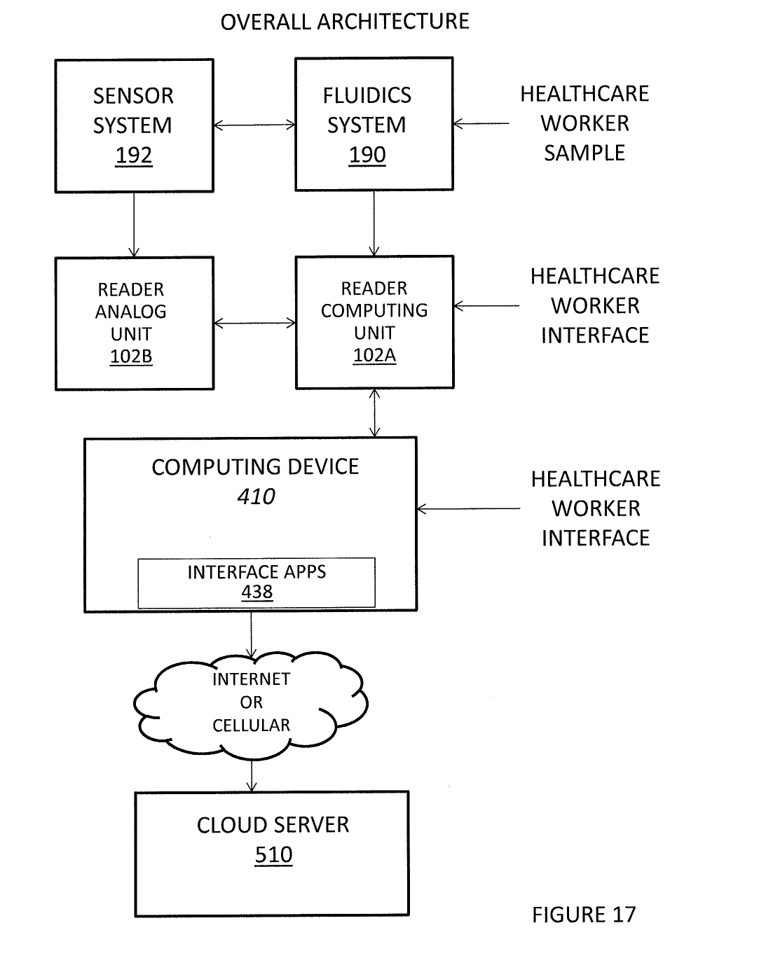

FIG. 17 illustrates another example architecture involving a diagnostic device 100. In this example, the diagnostic device 100 includes a fluidics system 190, a sensor system 192, a reader computing unit 102A, a reader analog unit 102B, a computing device 410 including interface applications 438, a data communication network, and a cloud server computing device 510.

In this example, the fluidics system 190 receives a sample from a healthcare worker. The reader computing unit 102A receives inputs from the healthcare worker, such as to select a mode of operation, or other inputs.

The fluidics system 190 and sensor system 192 operate under the control of the reader computing unit 102A and the sample is evaluated by the sensor system 192 and the reader analog unit 102B.

Data communication occurs between the reader computing unit 102A and the computing device 410. Data communication also occurs between the computing device 410 and a cloud server 510 across a data communication network. Examples of such data communication are discussed herein.

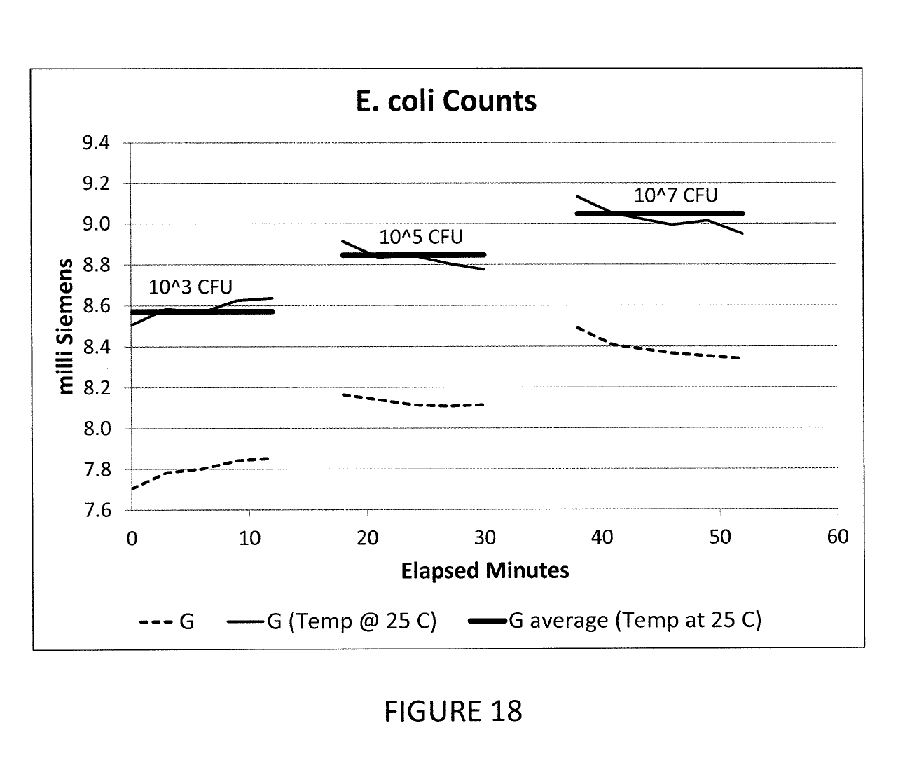

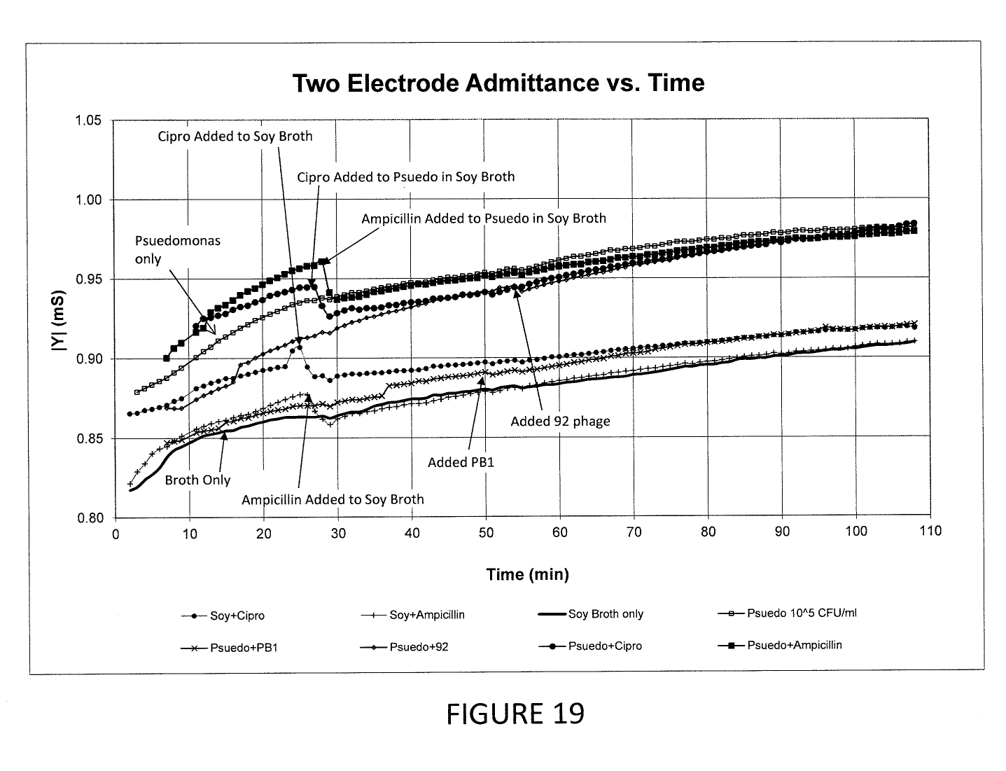

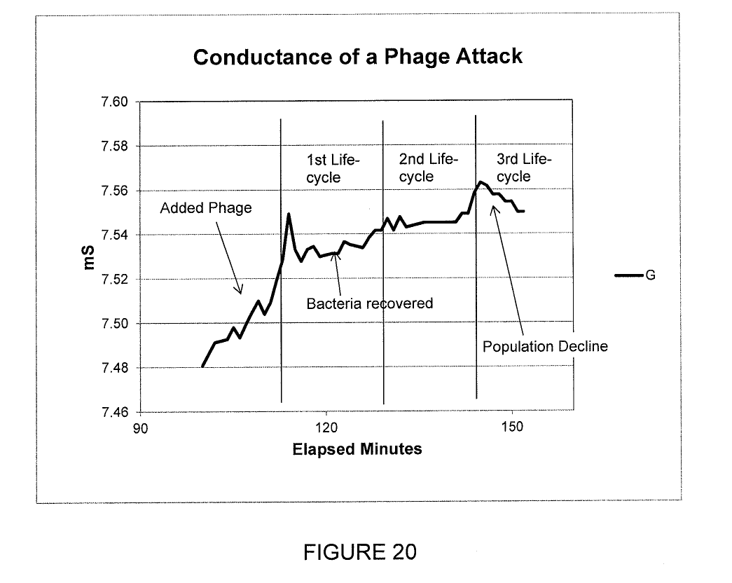

FIGS. 17-20 illustrate experimental data obtained using a diagnostic device 100.

The present disclosure uses the word “cell” in at least two contexts. One context is a biological “cell” and another context is a “sample cell.” To avoid confusion, a sample cell can alternatively be referred to as a sample unit, a test cell, a test unit, a sample module, or the like.

Some embodiments include one or more of the following:

An impedimetric measurement device for the monitoring of microbes present in a liquid medium comprising: an electrolytic solution, a containment vessel, two electrodes driven with an electric stimulus (forced electrodes) and two electrodes sensing an electric signal (sensed electrodes): a. For the use in monitoring the count of live microbes as a function of time; b. For the use in determining antibiotic sensitivity of the microbes; and/or c. For the use in identifying microbes using selected antimicrobials

A device with the electric stimulus being an ac voltage or current.

A device with the sensed electric signal being an ac voltage.

A device with the sensed electrodes located along the path of electrical signal current that flows between the forced electrodes.

A device with electrodes arranged in a four-cornered geometrical shape so that the forced electrodes are adjacent along the geometrical shape boundary and the sensed electrodes are adjacent along the geometrical shape boundary.

A device with the shape of the electrolyte containment vessel boundary generalized to encompass the electrodes while directing the electric field for optimal performance.

A device with the electrodes fabricated on a planar substrate comprising the bottom of the electrolytic cell.

A device with a printed circuit board comprising the planar substrate.

A device of claim 6 with the electrodes installed within the side walls of the electrolytic cell, and at or adjacent a bottom of the electrolytic cell.

A device wherein a volume is defined to locate an antimicrobial impregnated material for the purpose of introducing a measured amount of one or a plurality of antimicrobial agents into the cell, when the cell is filled with an electrolytic solution.

A device calibrated to provide indications of microbial concentrations.

An algorithm for detection of increasing and decreasing microbial concentrations as a function of time.

An algorithm with temperature compensation of measurement data from multiple cells and referenced to cell containing only an electrolytic solution.

An algorithm with capability to compare data from individual cells, for the purpose of indicating relative biological activity within such cells.

An algorithm with capability of discriminating the effectiveness of an antimicrobial agent present within a particular cell.

Any of the algorithms described herein, wherein the algorithm is performed by or using a computing device.

A device that has a volume defined that holds a material impregnated with a measured concentration of one or more bacteriophage which when wetted releases the bacteriophage into the electrolytic solution.

A device that has a volume defined that holds a material impregnated with one or more antimicrobials which when wetted releases the antimicrobial into the electrolytic solution.

A method wherein the sensed voltage is used to calculate conductance or admittance.

A device whose shape alleviates cell factor dependence on the containment vessels fill factor.

A device with a containment vessel comprising: a. at least one substrate; b. at least four electrodes; and c. a sample cavity formed in the at least one substrate, the sample cavity comprising: i. a sensing portion including the electrodes therein, the sensing portion having a shape configured to direct and focus electric fields generated by the electrodes within the sample cavity; and ii. a shape extending from the sensing portion and having a cross-sectional size that is less than a cross-sectional size of the sensing portion, the shape extending having a volume, wherein a volume of the extending portion permits a volume of the sample to vary without substantially affecting electrical measurements from the electrodes.

A method of using the conductance of the broth only sample cavity to correct all of the signatures for temperature variation during the duration of the test.

Means for keeping the liquid in all of the sample cavities at the same temperature during the test.

Means for heating the sample holder to a temperature of 35 degrees C. if that is required to achieve more robust admittance and conductance signatures.

Using an A/D converter to reduce the analog AC currents and voltages to a digital format that can be used to more easily calculate and compare the admittance and conductance signatures.

A diagnostic device including one of the various possible sample cavity and electrode geometries and the required mechanical tolerances of electrode size and spacing and how those affect the variation in the calibration factor for the plurality of sample cavities in a sample holder.

A method of using the four-terminal measurement of conductance to achieve temperature compensation from the broth only sample cavity and to avoid the negative effects of biofilm growth on the electrodes.

A method of operating a diagnostic device by defining the limits for the applied alternating current and/or voltage to avoid plating effects and electrolysis at the electrode surfaces.

A method of defining biocompatible materials used in the construction of the antimicrobial dispenser.

A method of providing viral phages that are specific to each type bacteria and using the viral phages to identify the bacteria.

A method of utilizing the unique conductance signature of an effective phage attack.

A diagnostic device utilizing a nutrient broth that promotes bacterial growth and has a controlled conductivity and temperature coefficient.

A method of identifying the microbes present in a sample involving identifying the ratio of admittance and/or conductance signatures between the broth+bacteria, and broth+bacteria+viral phage sample cavities.

A method of identifying an effective antimicrobial for the identified bacterium involving determining the ratio of admittance and/or conductance signatures between the broth+bacteria and broth+bacteria+antimicrobial sample cavities.

A method of determining the CFU concentration of the bacteria in a sample involving identifying a conductance signature of the broth+bacteria sample cavity.

Means for heating the sample holder to a temperature of 35 degrees C.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

1. A diagnostic device comprising:
at least one sample module defining a sample cavity therein;
at least four electrodes arranged in the sample cavity; and
electronic circuitry operably connected to the electrodes, wherein the electronic circuitry is operable in a first mode and a second mode, wherein when operating in the first mode, the electronic circuitry operates to determine a conductance of a sample in the sample cavity, and wherein when operating in the second mode, the electronic circuitry operates to determine an admittance of the sample in the sample cavity.

2. The diagnostic device of claim 1, wherein the diagnostic device operates to identify a microbe in the sample.

3. The diagnostic device of claim 2, wherein the microbes are selected from bacteria, fungi, viruses, or nematodes.

4. The diagnostic device of claim 1, wherein the diagnostic device operates to count microbes in the sample.

5. The diagnostic device of claim 1, wherein the diagnostic device determines an antimicrobial sensitivity of a microbial in the sample.

6. The diagnostic device of claim 5, wherein the sample module further includes an antimicrobial agent selected from a bacteriophage, a mycovirus, a virophage, a nematophage, an antibiotic, an antimicrobial, an antiviral, an antifungal, a parasiticide, or any combination thereof.

7. (canceled)

8. A sample module comprising:
at least one substrate;
at least four electrodes; and
a sample cavity formed in the at least one substrate, the sample cavity comprising:
a sensing portion including the electrodes therein, the sensing portion having a shape configured to direct and focus electric fields generated by the electrodes within the sample cavity; and
a chimney portion extending from the sensing portion and having a cross-sectional size that is less than a cross-sectional size of the sensing portion.

9. The sample module of claim 8, wherein the shape and position of the chimney portion permits variation in a volume of the sample while storing an excess portion of the sample outside of the sample cavity, whereby the variation in the volume of the sample does not substantially affect electrical measurements at the electrodes.

10. A diagnostic device comprising:
a plurality of sample modules;
electrodes arranged in the sample modules;
a calibration fluid disposed in a calibration module of the sample modules; and
electronic components coupled to the electrodes, wherein the electronic components are operable to measure a conductivity of the fluid in the calibration cell and to determine a temperature of the calibration fluid using the measured conductivity.
